(19) United States
(12) Patent Application Publication
(10) Pub. No.: US 2024/0374456 A1
(43) Pub. Date: Nov. 14, 2024

(54) MODEL GENERATION METHOD, MODEL GENERATION DEVICE, PHASE ESTIMATION METHOD, CONTROL METHOD, AND CONTROL DEVICE

(71) Applicant: ADVANCED TELECOMMUNICATIONS RESEARCH INSTITUTE INTERNATIONAL, Soraku-gun, Kyoto (JP)

(72) Inventors: Tomoyuki NODA, Soraku-gun (JP); Tatsuya TERAMAE, Soraku-gun (JP)

(73) Assignee: ADVANCED TELECOMMUNICATIONS RESEARCH INSTITUTE INTERNATIONAL, Soraku-gun, Kyoto (JP)

(21) Appl. No.: 18/690,614

(22) PCT Filed: Aug. 3, 2023
(86) PCT No.: PCT/JP2023/028479

§ 371 Date: Mar. 8, 2024

(30) Foreign Application Priority Data
Aug. 5, 2022 (JP) 2022-125530

(51) Int. Cl. A61H 3/00

(52) U.S. Cl. CPC A61H 3/008; A61H 2201/1207; A61H 2201/1409; A61H 2201/1642; A61H 2201/5007; A61H 2201/5061; A61H 2201/5064; A61H 2201/5071; A61H 2201/5084; A61H 2203/0487; A61H 2230/625

(57) ABSTRACT

A computer acquires sensor data generated by measuring walking of one or more cycles of a user with a sensor, calculates an estimated value of a gait phase in the sensor data using a reference model, calculates an ideal value of the phase corresponding to the calculated estimated value based on a period of the walking that appears in the sensor data, and generates the correction model by modeling an error between the estimated value of the gait phase and the ideal value. This provides a technique for estimating the gait phase easily and accurately in real time.

### TECHNICAL FIELD

The present invention relates to a model generation method, a model generation device, a phase estimation method, a control method, and a control device.

### BACKGROUND ART

For example, in a situation where rehabilitation is performed, the phase in a gait cycle is estimated in real time in some cases in order to assist gait according to a pattern. In general, the phase of gait cycle is defined by a continuous value obtained by normalizing the time of a gait cycle in the range of 0 to 2π. Analyzing the gait cycle after walking motion is performed is easy because it only requires dividing the amount of time from one heel strike to the next heel strike. On the other hand, it is not easy to estimate the gait phase in real time because there are individual differences in human gait. In view of this, in conventional methods, the phase of gait cycle is estimated using a model that approximates human gait (for example, PTL 1 and PTL 2).

### CITATION LIST

### Patent Literature

[Patent Literature 1] Japanese Patent Application Publication No. 2017-217039
[Patent Literature 2] Japanese Patent Application Publication No. 2015-008960

### SUMMARY OF THE INVENTION

### Technical Problem

According to the conventional method, the amount of calculation required for phase estimation can be reduced by using an approximation model. However, the inventor of the present invention found that the conventional method has the following problem. That is, since the conventional method uses an approximation model, an error occurs between the estimated value and the true value of the phase.

FIG. 1 shows an example of errors that occur when using an approximation model. As described above, the true value of the phase of the gait cycle can be calculated by dividing the amount of time from one heel strike to the next heel strike. The error in FIG. 1 is obtained by associating the true value obtained through this method with the output (estimated value of the phase) of the approximation model at each sampling time. Note that the data in FIG. 1 was obtained under the same conditions as the experimental examples described later.

In the phase space of FIG. 1, it is ideal that the output of the approximation model is a straight line with respect to the true value in the range of 0 to 2π. However, there are individual differences in how people walk. For this reason, it is difficult to adjust the parameters of the approximation model so that no error occurs. As shown in FIG. 1, the output of the approximation model is distorted with respect to the true value. Also, if approximation models are created individually, the cost will increase. Accordingly, with conventional methods, it has been difficult to estimate the gait phase easily and accurately in real time.

Note that various problems may arise due to the inability to accurately estimate the phase of gait cycles. In one example, in the above-described situation where gait assistance is performed, a skilled physical therapist defines and optimizes a gait assistance pattern in a phase space. If an error occurs in the estimated phase, the time-series pattern of the assistance that is executed will change. That is, it becomes difficult to perform gait assistance as intended by the physical therapist. On the other hand, there is a method that provides assistance based on the assumption that the walking speed is a certain amount. However, with this method, the timing when gait assistance is activated is too early or too late for patients with unstable gait, such as patients suffering from after-stroke paralysis, and thus it is difficult to appropriately assist users whose walking speed or stride length tends to change. Accordingly, in order to realize gait assistance at an appropriate timing, there is a need for a technique by which it is possible to easily and accurately estimate the gait phase in real time.

One aspect of the present invention has been made in consideration of such points, and an object thereof is to provide a technique for estimating the gait phase easily and accurately in real time.

### Solution to Problem

The present invention adopts the following configuration in order to solve the above-mentioned problems. Note that the configurations of the invention described below can be combined as appropriate.

A model generation method according to one aspect of the present invention is a model generation method to be executed by a computer, including the steps of: acquiring sensor data generated by measuring walking of one or more cycles of a user with a sensor; calculating an estimated value of a gait phase in the acquired sensor data, using a reference model; calculating an ideal value of the gait phase corresponding to the calculated estimated value, based on a cycle of the walking that appears in the sensor data; and generating a correction model by modeling an error between the estimated value of the gait phase and the ideal value.

In this configuration, generating the correction model is merely modeling the error between the estimated value of the phase of the obtained sensor data and the ideal value. As described above, after walking is performed, it is easy to calculate the true value (ideal value) of the gait phase. For this reason, it is possible to easily generate a correction model for each user. Also, it is easy to generate a correction model again depending on circumstances such as the user's status, such that the phase can be estimated with high accuracy. Furthermore, since the generated correction model is merely configured to show the correspondence between the estimated value of the reference model and the error, calculation of the correction model is easy. Accordingly, with this configuration, the gait phase can be estimated easily and accurately in real time using the generated correction model.

In the model generation method according to the above aspect, the sensor data may be generated by measuring the walking of a plurality of cycles. After executing the step of calculating the estimated value of the phase, the computer may further execute the steps of: calculating variation in the calculated estimated value of the phase; and performing notification of an alert if a magnitude of the variation in the estimated value exceeds a threshold. If the estimated value of the phase varies in the obtained sensor data, the error between the estimated value and the ideal value also varies, and therefore there is a possibility that the accuracy of the generated correction model will deteriorate. On the other hand, according to the configuration, it is possible to alert that the accuracy of such a correction model may deteriorate.

In the model generation method related to the above-described aspects, the sensor data may be generated by measuring the walking while the user receives assistance. According to this configuration, the correction model can be generated in a situation where the user receives gait assistance. By using the generated correction model, the gait phase can be easily and accurately estimated in real time, and gait assistance can be performed at an appropriate timing.

In the model generation method related to the above-described aspects, the computer may repeatedly execute a generation cycle including the step of acquiring the sensor data, the step of calculating the estimated value of the phase, the step of calculating the ideal value of the phase, and the step of generating the correction model. According to this configuration, new correction models can be repeatedly generated by repeatedly executing the generation cycle. In one example, by generating a new correction model in response to changes in the user's walking motion, it is possible to continuously and accurately estimate the gait phase of the user.

In the model generation method according to the above-described aspect, in the step of calculating the estimated value of the phase in the second generation cycle or later, the computer may calculate the estimated value of the gait phase in the sensor data acquired in the current generation cycle by using a corrected reference model obtained by correcting a reference model used in the previous generation cycle using a correction model generated in the previous generation cycle. According to this configuration, by repeating the correction of the reference model using the correction model and the generation of the correction model for the corrected reference model, it is possible to obtain models (reference model, correction model) that can easily and accurately estimate the gait phase in real time.

In the model generation method related to the above-described aspects, after executing the generation cycle, the computer may execute the next generation cycle in response to a request from an operator. Due to the walking of the user changing over time, the generated correction model no longer matches the user in some cases. One example is a case where gait assistance (rehabilitation) is performed for a predetermined amount of time using a correction model. In this case, due to the walking of the user improving, there is a possibility that the correction model created before rehabilitation will no longer match the user and new errors will occur. With this configuration, in such a case, the correction model can be generated again in response to a request from an operator.

The mode of the present invention is not limited to the above-described model generation method. One aspect of the present invention may be an information processing method including a step of correcting an estimated value of a gait phase using a correction model generated through the model generation method according to any one of the above-described modes.

As an example, a phase estimation method according to one aspect of the present invention may be a phase estimation method to be executed by a computer, including the steps of: acquiring a sensor value of a sensor regarding walking of a user; calculating an estimated value of a gait phase from the acquired sensor value, using a reference model; estimating an error from the calculated estimated value of the phase, using a correction model; correcting the calculated estimated value of the phase using the estimated error; and outputting information relating to the corrected estimated value of the phase. The correction model may be generated by modeling the error between the estimated value of the gait phase and an ideal value, using training sensor data generated by measuring the walking of one or more cycles of the user with the sensor. The correction model may be generated by the model generation method according to any one of the above modes. According to this configuration, by using the correction model, the gait phase can be estimated easily and accurately in real time.

In the phase estimation method according to the above aspect, the computer may further execute the steps of: specifying a shift in the phase caused by a delay relating to estimation of the phase; and further correcting the corrected estimated value of the phase using the specified shift. Information relating to the corrected estimated value of the phase may be constituted by information relating to the further-corrected estimated value of the phase. According to the configuration, the influence of delay can be reduced.

Note that outputting information relating to the corrected estimated value of the phase may include outputting the corrected estimated value of the phase as-is (e.g., outputting audio, displaying an image, etc.). Also, outputting information relating to the corrected estimated value of the phase may also include executing information processing based on the obtained estimated value, and outputting the result of executing the information processing as information relating to the estimated value of the phase. The output of the result of executing the information processing may include controlling the operation of the controlled device according to the estimated value of the phase. The controlled device may be, for example, an intervention device such as a gait assistance device, an electrical stimulation device, or an activation measurement device.

As an example, a control method according to one aspect of the present invention may be a control method to be executed by a computer, including the steps of: acquiring a sensor value of a sensor regarding walking of a user; calculating an estimated value of a gait phase from the acquired sensor value, using a reference model; estimating an error from the calculated estimated value of the phase, using a correction model; correcting the calculated estimated value of the phase using the estimated error; determining a drive amount of a controlled device from the corrected estimated value of the phase; and outputting the determined drive amount. The correction model may be generated by modeling the error between the estimated value of the gait phase and an ideal value, using training sensor data generated by measuring the walking of one or more cycles of the user with the sensor. As a result, the controlled device can be driven at an appropriate timing according to the walking of the user.

In the control method according to the above aspect, the controlled device may be a gait assistance device, an electrical stimulation device, or an activation measurement device. Determining the drive amount of the controlled device may be determining the assistance amount of the gait assistance device, determining the amount of electrical stimulation by the electrical stimulation device, or determining the amount of electrical stimulation in the activation measurement device. Determining an amount may include determining whether or not to perform provision thereof.

In the control method according to the above-described aspect, the computer may further execute a step of specifying a shift in the phase caused by a delay relating to estimation of the phase, and a step of further correcting the corrected estimated value of the phase using the identified shift. Determining the drive amount of the controlled device from the corrected estimated value of the phase may be determining the drive amount of the controlled device from the further-corrected estimated value of the phase. As a result, the controlled device can be driven at a more appropriate timing.

As another example, a control method according to one aspect of the present invention may be an information processing method to be executed by a computer, including the steps of: setting an assistance pattern; acquiring a sensor value of a sensor regarding walking of a user; calculating an estimated value of a gait phase from the acquired sensor value, using a reference model; estimating an error from the calculated estimated value of the phase, using a correction model; correcting the calculated estimated value of the phase using the estimated error; determining an assistance amount of a gait assistance device from the corrected estimated value of the phase in accordance with the set assistance pattern; and outputting the determined assistance amount. The correction model may be generated by modeling the error between the estimated value of the gait phase and an ideal value, using training sensor data generated by measuring the walking of one or more cycles of the user with the sensor. The correction model may be generated by the model generation method according to any one of the above-described modes. According to this configuration, by using the correction model, the gait phase can be estimated easily and accurately in real time. As a result, gait assistance can be performed for the user at an appropriate timing.

In the control method according to the above-described aspect, the assistance pattern may be constituted by one or more muscle modules. The muscle module is configured by combining a plurality of periodic functions so as to reproduce muscular synergy. According to this configuration, due to the muscle module being configured as described above, it is possible to realize an assistance pattern that suits muscle synergy through simple calculation. In addition, by selecting one or more muscle modules, it is possible to easily create an assistance pattern that suits muscle synergy.

In the control method according to the above-described aspect, the gait assistance device may be configured to assist the walking through output of a pneumatic artificial muscle. Even if the assistance pattern is not smoothed, the assistance amount that is actually given will be smoothed due to the dynamics of the pneumatic artificial muscle (e.g., the delay of the motor driver, etc.). For this reason, according to this configuration, smooth assistance can be performed without incurring an increase in the calculation amount.

In the control method according to the above aspect, the computer may further execute the steps of: specifying a shift in the phase caused by a delay relating to estimation of the phase; and further correcting the corrected estimated value of the phase using the specified shift. Determining the assistance amount of the gait assistance device from the corrected estimated value of the phase may be determining the assistance amount of the gait assistance device from the further-corrected estimated value of the phase. This allows gait assistance to be performed at a more appropriate timing.

In the control method according to the above-described aspects, the computer may repeatedly execute an estimation cycle including the step of acquiring the sensor value, the step of calculating the estimated value of the phase, the step of estimating the error, the step of correcting the estimated value of the phase, the step of determining the assistance amount, and the step of outputting the assistance amount. In response to repeatedly executing the estimation cycle for the walking of one or more cycles of the user, the computer may further execute the steps of: calculating an ideal value of the gait phase with respect to the corrected estimated value, based on a cycle of the walking; calculating an error between the corrected estimated value and the ideal value; and outputting information relating to the calculated error. Due to the user repeatedly receiving gait assistance, the walking of the user changes in some cases (one example of a typical factor is that the walking of the user improves due to the assistance). According to this configuration, in such a case, it is possible to evaluate whether or not the correction model being used is starting to no longer match the user, by calculating the error between the corrected estimated value and the ideal value.

In the control method according to the above-described aspects, the computer may repeatedly execute an estimation cycle including the step of acquiring the sensor value, the step of calculating the estimated value of the phase, the step of estimating the error, the step of correcting the estimated value of the phase, the step of determining the assistance amount, and the step of outputting the assistance amount. In the step of determining the assistance amount in the second estimation cycle or later, the computer may calculate an amount of change between the corrected estimated value in the previous estimation cycle and the corrected estimated value in the current estimation cycle, determine whether or not the calculated amount of change satisfies a permissible condition, if the amount of change satisfies the permissible condition, determine an assistance amount in the current estimation cycle from the corrected estimated value in the current estimation cycle, and if the amount of change does not satisfy a permissible condition, determine the assistance amount in the current estimation cycle based on the corrected estimation value in the previous estimation cycle, without relying on the corrected estimation value in the current estimation cycle. During the repeated execution of phase estimation, the estimated value of the phase calculated from the sensor value changes suddenly or changes in a retrograde manner in some cases due to, for example, the user performing a walking motion that is different from the expected walking motion. According to this configuration, by determining whether or not the corrected estimated value of the phase satisfies a permissible condition, it is possible to monitor such unexpected behavior of the estimated value of the phase. Then, if the corrected estimated value of the phase changes such that it does not satisfy the permissible condition, appropriate assistance can be performed by using the estimation result of the previous estimation cycle instead of using that estimated value.

In another mode of each of the model generation method, phase estimation method, and control method according to the above-described aspects, one aspect of the present invention may also be an information processing device configured to realize all or part of each of the above-described configurations, or a storage medium readable by a computer, another apparatus or device, or the like, storing such a program. Here, a storage medium readable by a computer or the like is a medium that stores information such as programs through electrical, magnetic, optical, mechanical, or chemical action.

For example, the model generation device according to one aspect of the present invention may include: a data acquisition unit configured to acquire sensor data generated by using a sensor to measure walking of one or more cycles of a user; a phase estimation unit configured to calculate an estimated value of a gait phase in the acquired sensor data, using a reference model; a calculation unit configured to calculate an ideal value of the gait phase corresponding to the calculated estimated value based on a cycle of the walking that appears in the sensor data; and a generation unit configured to generate a correction model by modeling an error between the estimated value of the gait phase and the ideal value.

Also, for example, the phase estimation device according to one aspect of the present invention may include: an acquisition unit configured to acquire a sensor value of a sensor regarding walking of a user; a phase estimation unit configured to calculate an estimated value of the gait phase from the acquired sensor value; an error estimation unit configured to estimate an error from the calculated estimated value of the phase using a correction model; a correction unit configured to correct the calculated estimated value of the phase based on the estimated error; and an output unit configured to output information relating to the corrected estimated value of the phase.

Also, for example, a control device according to one aspect of the present invention may include: a setting unit configured to set an assistance pattern; an acquisition unit configured to acquire a sensor value of a sensor regarding walking of a user; a phase estimation unit configured to calculate an estimated value of a gait phase from the acquired sensor data, using a reference model; an error estimation unit configured to estimate an error from the calculated estimated value of the phase, using a correction model; a correction unit configured to correct the calculated estimated value of the phase using the estimated error; and an output unit configured to determine an assistance amount from the corrected estimated value of the phase in accordance with the set assistance pattern and output the determined assistance amount.

### Advantageous Effects of Invention

According to the present invention, it is possible to provide a technique for estimating a gait phase easily and accurately in real time.

### BRIEF DESCRIPTION OF DRAWINGS

FIG. 1 shows an example of an error that occurs when using an approximation model.

FIG. 2 schematically shows an example of a situation in which the present invention is applied.

FIG. 3 schematically shows an example of a hardware configuration of a model generation device according to an embodiment.

FIG. 4 schematically shows an example of a hardware configuration of a phase estimation device according to an embodiment.

FIG. 5 schematically shows an example of a software configuration of a model generation device according to an embodiment.

FIG. 6 schematically shows an example of a software configuration of a phase estimation device according to an embodiment.

FIG. 7 is a flowchart illustrating an example of a processing procedure of a model generation device according to an embodiment.

FIG. 8 shows an example of a correction model according to an embodiment.

FIG. 9 is a flowchart illustrating an example of a processing procedure of a phase estimation device according to an embodiment.

FIG. 10A schematically shows an example of a process of processing for calculating a corrected estimated value of a phase, using the correction model according to the embodiment.

FIG. 10B shows an example of estimated values of a phase obtained before and after correction by a correction model according to an embodiment.

FIG. 11 schematically shows an example of another situation (control device of a gait assistance device) in which the present invention is applied.

FIG. 12 schematically shows an example of a hardware configuration of a control device according to a modified example.

FIG. 13 schematically shows an example of a software configuration of a control device according to a modified example.

FIG. 14 is a flowchart illustrating an example of a processing procedure of a control device according to a modified example.

FIG. 15 schematically shows an example of a muscle module that forms an assistance pattern in a control device according to a modified example.

FIG. 16 schematically shows an example of another situation (control device for an electrical stimulation device) in which the present invention is applied.

FIG. 17 schematically shows an example of another situation (control device of an activation measurement device) in which the present invention is applied.

FIG. 18 schematically shows an example of another situation (gait abnormality monitoring device) in which the present invention is applied.

FIG. 19 is a diagram for illustrating a delay relating to phase estimation.

FIG. 20 schematically shows another example of a process of processing for calculating a corrected estimated value of the phase using the correction model according to an embodiment.

FIG. 21A shows the results of a real-time phase estimation test for a first test subject.

FIG. 21B shows the results of a real-time phase estimation test for a second test subject.

### DESCRIPTION OF EMBODIMENTS

An embodiment (hereinafter also referred to as “this embodiment”) according to an aspect of the present invention will be described below with reference to the drawings.

However, this embodiment described below is merely an illustration of the present invention in all respects. It goes without saying that various improvements and modifications can be made without departing from the scope of the invention. That is, in implementing the present invention, specific configurations according to the embodiments may also be adopted as appropriate. Note that although the data that appears in this embodiment is described using natural language, more specifically, it is designated using a quasi-language, commands, parameters, machine language, and the like that can be recognized by a computer.

### § 1 Application Example

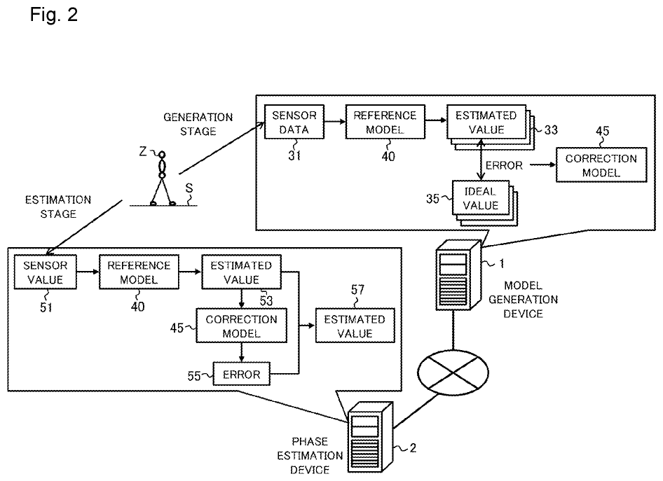

FIG. 2 schematically shows an example of a situation in which the present invention is applied. As shown in FIG. 2, the estimation system according to this embodiment includes a model generation device 1 and a phase estimation device 2.

### [Generation Stage]

The model generation device 1 according to the present embodiment is one or more computers configured to generate a correction model 45 for a user Z. In the present embodiment, the model generation device 1 acquires sensor data 31 generated by measuring walking of one or more cycles of the user Z with a sensor S. The model generation device 1 calculates an estimated value 33 of the gait phase of the walking in the acquired sensor data 31, using the reference model 40. The model generation device 1 calculates an ideal value (true value) 35 of the gait phase with respect to the calculated estimated value 33 based on the cycle of walking that appears in the sensor data 31. The model generation device 1 generates a correction model 45 by modeling an error between the estimated value 33 and the ideal value 35 of the gait phase. The generated correction model 45 may be provided to the phase estimation device 2 at any timing.

### [Estimation Stage]

On the other hand, the phase estimation device 2 according to the present embodiment is one or more computers configured to estimate the gait phase of the user Z in real time using the reference model 40 and the correction model 45. In this embodiment, the phase estimation device 2 acquires a sensor value 51 of the sensor S regarding the walking of the user Z. The phase estimation device 2 calculates an estimated value 53 of the gait phase from the acquired sensor value 51, using the reference model 40. The phase estimation device 2 estimates an error 55 from the calculated estimated value 53 of the phase using the correction model 45. The correction model 45 is generated by the model generation device 1 by modeling the error between the estimated value 33 and the ideal value 35 of the gait phase, using the sensor data 31 for training. The phase estimation device 2 corrects the calculated estimated value 53 of the phase using the estimated error 55. As a result, the phase estimation device 2 obtains a corrected estimated value 57 of the phase. The phase estimation device 2 outputs information relating to the corrected estimated value 57 of the phase. Note that in this embodiment, for convenience of description, the stage in which the correction model 45 is generated by the model generation device 1 is referred to as a generation stage, and the stage in which the phase estimation device 2 estimates the gait phase is referred to as an estimation stage.

### [Characteristics]

As described above, in this embodiment, generating the correction model 45 is merely modeling the error between the estimation result (estimated value 33) of the reference model 40 and the true value (ideal value 35) using the sensor data 31. On the basis of a cycle of walking that appears in the sensor data 31, it is easy to calculate the ideal value 35 (true value) of the gait phase. For this reason, the correction model 45 can be easily created for each user Z. Also, it is easy to generate the correction model 45 again according to circumstances such as the status of the user z, such that the gait phase can be estimated with high accuracy. Furthermore, since the generated correction model 45 is merely configured to show the correspondence between the estimated value and the error of the reference model 40 (that is, calculate the error from the estimated value), the calculation of the correction model 45 is easy. Accordingly, with the model generation device 1 according to the present embodiment, it is possible to generate a correction model 45 that corrects the output of the reference model 40 and enables the gait phase to be estimated easily and accurately in real time. In the phase estimation device 2 according to this embodiment, by using such a correction model 45 together with the reference model 40, it is possible to easily and accurately estimate the gait phase of the user Z in real time.

### (Sensor)

Note that there is no particular limitation on the type of the sensor S as long as it can capture the walking motion of a person (user Z), and the sensor S may be selected as appropriate according to the embodiment. The sensor S may be, for example, a plantar sensor, an image capture device, a motion capture device, a myoelectric sensor, an acceleration sensor, a gyro sensor, a pressure distribution sensor, a combination thereof, or the like. The sensor S may be constituted by a plurality of types of sensors. The sole is the surface of the foot that comes into contact with the ground. A plantar sensor is configured to measure forces acting from a surface of a person's foot when walking. The plantar sensor may include, for example, a load sensor, a force sensing resistor, a load cell, a capacitive force sensor, or the like.

The sensor data 31 used in the generation stage may be obtained by measuring one or more walking motions performed by the user Z using the sensor S. In a typical example, the sensor data 31 may be configured to include a plurality of sensor values for walking of one cycle by measuring the walking of the user Z at any sampling interval. The number of sensor values included in the sensor data 31 may be determined as appropriate according to the measurement time, sampling interval, and the like. On the other hand, in the estimation stage, the sensor value 51 may be obtained by measuring the walking motion of the user Z using the sensor S at the timing of estimating the gait phase. The gait phase of the user Z may be estimated in real time by executing the estimation processing in response to obtaining the sensor value 51.

The timing of acquiring the sensor data 31 need not be particularly limited, and may be determined as appropriate according to the embodiment. For example, the sensor data 31 may be obtained by measuring the walking motion of the user Z using the sensor S, separately from the processing in the estimation stage. Also, in the estimation stage, the sensor data 31 may be acquired at the same time as the processing for estimating the gait phase is executed. In this case, at least some of the sensor data 31 may be constituted by a plurality of sensor values 51 obtained while repeating the estimation processing.

### (Reference Model)

The reference model 40 is configured to perform calculation processing for calculating an estimated value of a gait phase from the sensor value of the sensor S. The configuration of the reference model 40 need not be particularly limited as long as it is a calculation model that can perform such calculation processing, and the reference model 40 may be determined as appropriate according to the embodiment. The reference model 40 may be configured to accept input of a sensor value based on, for example, a data table, a function formula, a rule, or the like, and calculate an estimated value of the phase from the input sensor value. In a typical example, the reference model 40 may be an approximation model illustrated in PTL 1, 2, and the like. The method proposed in the reference literature (Tomoyuki Noda, Tatsuya Teramae, Asuka Takai, Kimitaka Hase, Jun Morimoto, “Robotization of everyday orthotics: Development of a short leg orthosis with modular joints driven by pneumatic artificial muscles” MB Medical Rehabilitation No. 205:22-27, 2017, <3. Phase synchronization control with walking and assistance test>) may be adopted in the reference model 40. In addition, the method proposed in the reference literature (Luka Peternel, Tomoyuki Noda, Tadej Petric, Ales Ude, Jun Morimoto, Jan Babic, “Adaptive Control of Exoskeleton Robots for Periodic Assistive Behaviours Based on EMG Feedback Minimisation” [online], [retrieved Mar. 28, 2022], Internet <URL: https://journals.plos.org/plosone/article/authors?id=10.1371/journal.pone.0148942>) may also be adopted in the reference model 40. In addition, the reference model 40 may be constituted by a trained machine learning model generated through machine learning. The machine learning model may be constituted by, for example, a neural network, a support vector machine, a regression model, or the like.

### (Ideal Value)

The ideal value 35 (true value) of the gait phase may be calculated by a posteriori analysis of the cycle of walking represented by the sensor data 31, in correspondence with each sensor value included in the sensor data 31. In one example, the ideal value 35 may be calculated by equally dividing the phase with respect to the time from one heel strike to the next heel strike appearing in the sensor data 31. In another example, the ideal value 35 may be calculated by equally dividing the phase with respect to the time during which the estimated value obtained by the reference model 40 for the time is 0 to 2π (one cycle of walking). In either method, the ideal value 35 can be easily calculated.

### (Correction Model)

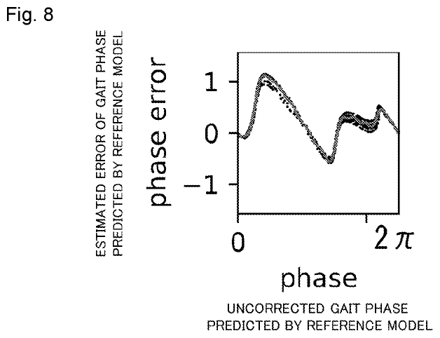

The correction model 45 may be generated by associating the estimated value 33 and the ideal value 35 of the phase at each sampling time and modeling the error between the estimated value 33 and the ideal value 35. As a result, the correction model 45 may be configured to execute calculation processing for calculating an error with respect to the estimated value of the gait phase from the estimated value. The configuration of the correction model 45 need not be particularly limited as long as it is a calculation model that can execute such calculation processing, and may be determined as appropriate depending on the embodiment. The correction model 45 may be configured to receive an input of an estimated value of the phase using a data table, a function formula, a rule, or the like, and calculate an error for the input estimated value (an example thereof is shown in FIG. 8, which will be described later). The correction model 45 may also be constituted by a machine learning model. As the method for generating the correction model 45, any method such as a method of simply modeling error, fitting, or machine learning may be used.

### (Output Processing)

The mode and content of the output processing of information relating to the corrected estimated value 57 of the phase may be determined as appropriate according to the embodiment. For example, outputting information relating to the corrected estimated value 57 of the phase may include outputting the corrected estimated value 57 of the phase as-is (e.g., outputting audio, displaying an image, etc.). Also, outputting information relating to the corrected estimated value 57 of the phase may include executing information processing based on the obtained estimated value 57, and outputting the execution result of the information processing as information relating to the estimated value 57 of the phase. Outputting the execution result of the information processing may include controlling the operation of the controlled device according to the estimated value 57 of the phase. The controlled device may be, for example, an intervention device such as a gait assistance device, an electrical stimulation device, or an activation measurement device. In one example, the phase estimation device 2 may be configured to operate as a control device of the gait assistance device, and control the gait assistance operation performed by the gait assistance device according to the corrected estimated value 57 of the phase in accordance with a set assistance pattern.

### (Generation Stage/Estimation Stage)

The generation processing performed by the model generation device 1 and the gait phase estimation processing performed by the phase estimation device 2 may be executed at any timings. In a typical example, the model generation device 1 may generate the correction model 45 as preprocessing. Thereafter, the phase estimation device 2 may use the generated correction model 45 to calculate the corrected estimated value 57 of the phase. In this case, the sensor data 31 may be acquired separately from the processing of the estimation stage.

In this embodiment, the model generation device 1 repeatedly executes a generation cycle including a step of acquiring the sensor data 31, a step of calculating the estimated value 33 of the phase, a step of calculating the ideal value 35 of the phase, and a step of generating the correction model 45. The phase estimation device 2 may repeatedly execute an estimation cycle including a step of acquiring the sensor value 51, a step of calculating the estimated value 53 of the phase, a step of estimating an error 55, a step of correcting the estimated value 53 of the phase (obtaining a corrected estimated value 57), and a step of outputting information relating to the corrected estimated value 53 of the phase.

After the phase estimation device 2 repeatedly executes processing for estimating the gait phase, the model generation device 1 may generate the correction model 45. In this case, at least some of the sensor data 31 may be collected while the phase estimation device 2 is estimating the gait phase. That is, at least some of the sensor data 31 may be constituted by a plurality of sensor values 51 obtained while repeating the estimation processing. Note that if the correction model 45 has already been generated, the phase estimation device 2 may estimate the gait phase using the reference model 40 and the correction model 45, and the model generation device 1 may generate a new correction model 45. If the correction model 45 has not been generated, the phase estimation device 2 may estimate the gait phase using only the reference model 40. That is, the phase estimation device 2 may execute the processing from when the sensor value 51 is acquired up to when the estimated value 53 is calculated.

In addition, the model generation device 1 may repeatedly execute the generation cycle regardless of the processing of the estimation stage. Also, the generation processing performed by the model generation device 1 and the estimation processing performed by the phase estimation device 2 may be repeatedly executed alternatingly. As a result, the generation of the correction model 45 performed by the model generation device 1 may be repeated such that the accuracy of the estimation processing performed by the phase estimation device 2 is ensured.

(User Status when Acquiring Sensor Data)

The status of the user Z when acquiring the sensor data 31 may be determined as appropriate according to the embodiment. As an example, a situation is envisioned in which the estimated value 57 of the phase obtained through the estimation processing is used to determine the amount of assistance of a gait assistance device. In this situation, the sensor data 31 may be generated by measuring walking while the user Z receives gait assistance. As a result, it is possible to generate a correction model 45 that enables accurate estimation of the gait phase in a situation where the user Z receives assistance from the gait assistance device.

In this situation of performing gait assistance, the model generation device 1 may generate the correction model 45 at any timing. In one example, the model generation device 1 may generate the correction model 45 as pre-processing performed when starting assistance performed by the gait assistance device. Also, the model generation device 1 may generate the correction model 45 according to an assistance pattern to be given to the user Z. For example, the assistance pattern may be changed by a physical therapist. The model generation device 1 may generate the correction model 45 as pre-processing performed when starting to assist the user Z in walking using the changed assistance pattern.

Also, in a situation where gait assistance is performed, the amount of gait assistance when acquiring the sensor data 31 may be determined as appropriate according to the embodiment. In one example, the amount of gait assistance when acquiring the sensor data 31 may be determined according to the estimated value of the gait phase, in accordance with a set assistance pattern. The estimated value of the gait phase may be obtained as a result of the estimation processing performed by the phase estimation device 2. If the correction model 45 has already been generated, the phase estimation device 2 may use the reference model 40 and the correction model 45 to calculate the estimated value 57 of the gait phase. The assistance amount may be determined according to the obtained estimated value 57. On the other hand, if the correction model 45 has not been generated, the phase estimation device 2 may use only the reference model 40 to calculate the estimated value 53 of the gait phase. The assistance amount may be determined according to the obtained estimated value 53.

However, the status of the user Z when acquiring the sensor data 31 need not be limited to such an example. In another example, the sensor data 31 may be generated by measuring walking of the user Z without assistance. In yet another example, the sensor data 31 may be generated by measuring the walking of the user Z using the sensor S while the user Z is receiving another intervention (for example, electrical stimulation).

### (Case of Repeating Generation Cycle)

In one example, if the generation cycle is repeatedly executed, in the second generation cycle and onward, the model generation device 1 may correct the reference model 40 using the correction model 45 generated in the previous generation cycle. As a result, the model generation device 1 may generate the corrected reference model 40 (that is, the model generation device 1 may update the reference model 40). In this case, in the step of calculating the estimated value 33 of the phase in the second generation cycle and onward, the model generation device 1 may use the generated corrected reference model 40 to calculate the estimated value 33 of the gait phase in the sensor data 31 acquired in the current generation cycle. Then, the model generation device 1 may execute the step of calculating the ideal value 35 of the phase and the step of generating the correction model 45, and generate a new correction model 45 for the corrected reference model 40. This makes it possible to obtain models (reference model 40, correction model 45) that can easily and accurately estimate the gait phase in real time.

However, the mode in which the generation cycle is repeated need not be limited to such an example. In another example, when repeatedly executing the generation cycle, the model generation device 1 may generate the correction model 45 again using the reference model 40 as-is. That is, the model generation device 1 may repeat the processing of the generation cycle and update the correction model 45 without updating the reference model 40. This mode may also be adopted in the situation where the above-mentioned gait assistance is performed. In this case, when acquiring the sensor data 31, the estimated value 57 of the gait phase may be calculated using the reference model 40 and the correction model 45 generated in the previous generation cycle, and the assistance amount may be determined according to the calculated estimated value 57. The sensor data 31 may be acquired while receiving gait assistance of the amount of assistance determined thereby. On the other hand, in the current generation cycle, the model generation device 1 may use only the reference model 40 to calculate the estimated value 33 of the gait phase in the sensor data 31, and generate a new correction model 45 for the reference model 40.

### (Handling Elapse of Time)

The sensor data 31 is acquired every time walking of the user Z is measured by the sensor S, and may be stored in any storage region. The model generation device 1 may generate the correction model 45 using at least some of the sensor data 31 that has been acquired up to the point in time when the generation cycle is executed. In one example, the model generation device 1 may generate the correction model 45 using all of the sensor data 31.

However, the walking motion of the user Z may change over time. When the walking motion of the user Z changes, the correction model 45 generated using the sensor data 31 acquired before the change may not match the walking motion of the user Z. That is, even if the correction model 45 is used, there is a possibility that the accuracy of estimating the gait phase of the user Z cannot be expected to improve. For this reason, it is preferable that the correction model 45 is generated and the sensor data 31 acquired closer to the point in time when the gait phase of the user Z is estimated is reflected more in the generation of the correction model 45.

In one example, the model generation device 1 may exclude the sensor data 31 after a predetermined period of time and generate the correction model 45 using the sensor data 31 within a predetermined period of time from the point in time when the generation cycle is executed. In another example, the model generation device 1 may weight the sensor data 31 according to the elapsed time such that the shorter the elapsed time is, the more that sensor data 31 is prioritized, and the longer the elapsed time is, the less that sensor data 31 is prioritized. The model generation device 1 may generate the correction model 45 using the weighted sensor data 31.

### (Device Configuration)

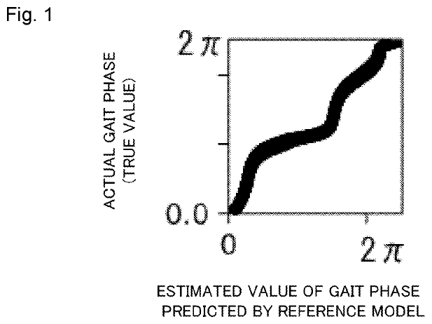

In one example, as shown in FIG. 1, the model generation device 1 and the phase estimation device 2 may be connected to each other via a network. The type of network may be selected as appropriate from, for example, the Internet, a wireless communication network, a mobile communication network, a telephone network, a dedicated network, and the like. However, the method for exchanging data between the model generation device 1 and the phase estimation device 2 need not be limited to such an example, and may be selected as appropriate according to the embodiment. In another example, data may be exchanged between the model generation device 1 and the phase estimation device 2 using a storage medium.

Also, in the example of FIG. 1, the model generation device 1 and the phase estimation device 2 are separate computers. However, the configuration of the system according to this embodiment need not be limited to such an example, and may be determined as appropriate according to the embodiment. In another example, the model generation device 1 and the phase estimation device 2 may be constituted by an integrated computer. In this case, the computer may generate the correction model 45 by operating as the model generation device 1. The generated correction model 45 may be used immediately in the operation as the phase estimation device 2. By operating as the phase estimation device 2, the computer may estimate the gait phase of the user Z using the reference model 40 and the correction model 45. The computer may switch between and execute the operations of the model generation device 1 and the phase estimation device 2 according to an operator's instructions or the like.

### § 2 Configuration Example

### [Hardware Configuration]

### <Model Generation Device>

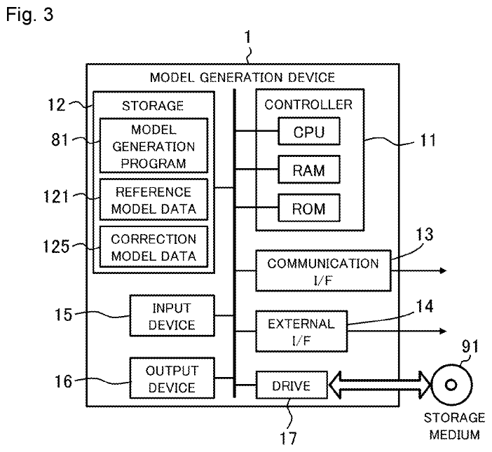

FIG. 3 schematically shows an example of the hardware configuration of the model generation device 1 according to the present embodiment. In the example of FIG. 3, the model generation device 1 according to the present embodiment is a computer in which a controller 11, a storage 12, a communication interface 13, an external interface 14, an input device 15, an output device 16, and a drive 17 are electrically connected to each other.

The controller 11 includes a CPU (Central Processing Unit), which is a hardware processor, a RAM (Random Access Memory), a ROM (Read Only Memory), and the like, and is configured to execute information processing based on programs and various types of data. The controller 11 (CPU) is an example of a processor resource.

The storage 12 is constituted by, for example, a hard disk drive, a solid state drive, or the like. The storage 12 is an example of a memory resource. In this embodiment, the storage 12 stores various types of information such as a model generation program 81, reference model data 121, and correction model data 125.

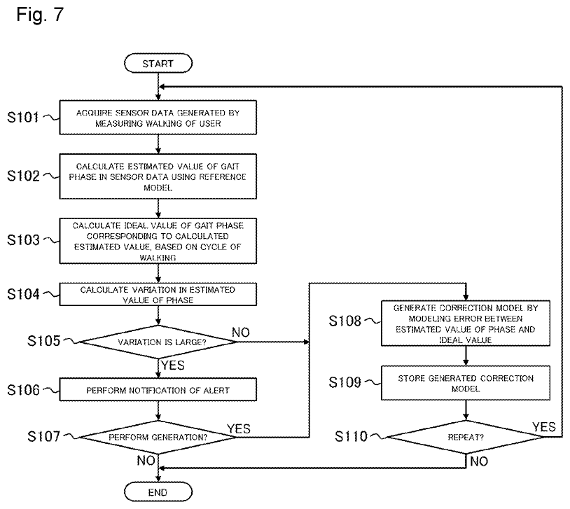

The model generation program 81 is a program for causing the model generation device 1 to execute information processing relating to generation of the correction model 45 (see FIG. 7, which will be described later). The model generation program 81 includes a series of instructions for the information processing. The reference model data 121 is configured to indicate information relating to the reference model 40. The correction model data 125 is configured to indicate information relating to the generated correction model 45. In this embodiment, the correction model data 125 is generated as a result of executing the model generation program 81.

The communication interface 13 is an interface for performing wired or wireless communication via a network. The communication interface 13 may be, for example, a wired LAN (Local Area Network) module, a wireless LAN module, or the like. The model generation device 1 may perform data communication with other computers via the communication interface 13.

The external interface 14 is an interface for connecting to an external device. The external interface 14 may be, for example, a USB (Universal Serial Bus) port, a dedicated port, or the like. The type and number of external interfaces 14 may be selected as appropriate.

The model generation device 1 may be connected to the sensor S for obtaining the sensor data 31 via the communication interface 13 or the external interface 14. When performing an intervention such as gait assistance on the user Z while acquiring the sensor data 31, the model generation device 1 may be connected to an intervention device (controlled device) such as a gait assistance device via the communication interface 13 or the external interface 14.

The input device 15 is a device for receiving input of information from an operator (e.g., a physical therapist, etc.). The input device 15 may be, for example, a mouse, a keyboard, or the like. The output device 16 is a device for outputting information to the operator. The output device 16 may be, for example, a display, a speaker, or the like. The operator can operate the model generation device 1 by using the input device 15 and the output device 16. The input device 15 and the output device 16 may be formed in one piece by, for example, a touch panel display or the like.

The drive 17 is a device for loading various types of information such as programs stored in the storage medium 91. The drive 17 may be, for example, a CD drive, a DVD drive, or the like. The storage medium 91 is a medium that accumulates information such as programs through electrical, magnetic, optical, mechanical, or chemical action such that computers, other devices, machines, and the like can read various types of information such as stored programs. At least one of the model generation program 81 and the reference model data 121 described above may be stored in the storage medium 91. The model generation device 1 may acquire at least one of the model generation program 81 and the reference model data 121 from the storage medium 91. Note that in FIG. 3, a disk-type storage medium such as a CD or a DVD is illustrated as an example of the storage medium 91. However, the type of the storage medium 91 is not limited to the disk type, and may be a type other than the disk type. An example of a storage medium other than a disk type is a semiconductor memory such as a flash memory. The type of the drive 17 may be selected as appropriate according to the type of the storage medium 91.

Note that, regarding the specific hardware configuration of the model generation device 1, constituent elements may be omitted, replaced, or added as appropriate according to the embodiment. For example, the controller 11 may also include a plurality of hardware processors. The type of the hardware processors is not particularly limited and may be selected as appropriate according to the embodiment. The storage 12 may also be constituted by a RAM and ROM included in the controller 11. At least one of the communication interface 13, the external interface 14, the input device 15, the output device 16, and the drive 17 may also be omitted. The model generation device 1 may also be constituted by a plurality of computers. In this case, the hardware configurations of the computers may or may not match. Also, the model generation device 1 may be an information processing device designed exclusively for the provided service, or may be a general-purpose server device, a general-purpose PC (Personal Computer), a tablet PC, a mobile terminal, or the like.

### <Phase Estimation Device>

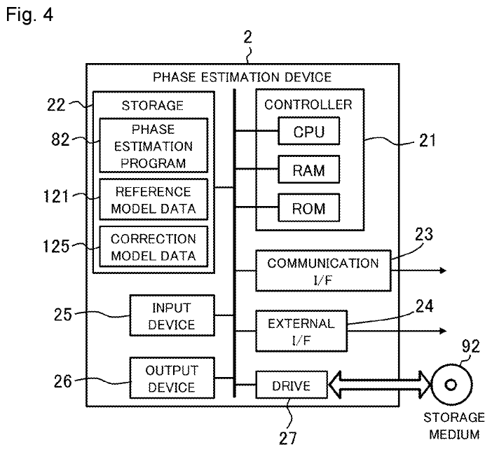

FIG. 4 schematically shows an example of the hardware configuration of the phase estimation device 2 according to this embodiment. In the example of FIG. 4, the phase estimation device 2 according to the present embodiment is a computer in which a controller 21, a storage 22, a communication interface 23, an external interface 24, an input device 25, an output device 26, and a drive 27 are electrically connected to each other.

The controller 21 to the drive 27 and the storage medium 92 of the phase estimation device 2 may be respectively configured similarly to the controller 11 to the drive 17 and the storage medium 91 of the model generation device 1. The controller 21 includes a CPU, a RAM, a ROM, and the like that are hardware processors, and is configured to execute various types of information processing based on programs and data. The controller 21 (CPU) is an example of a processor resource of the phase estimation device 2. The storage 22 is constituted by, for example, a hard disk drive, a solid state drive, or the like. The storage 22 is an example of a memory resource of the phase estimation device 2. In this embodiment, the storage 22 stores various types of information such as a phase estimation program 82, reference model data 121, and correction model data 125.

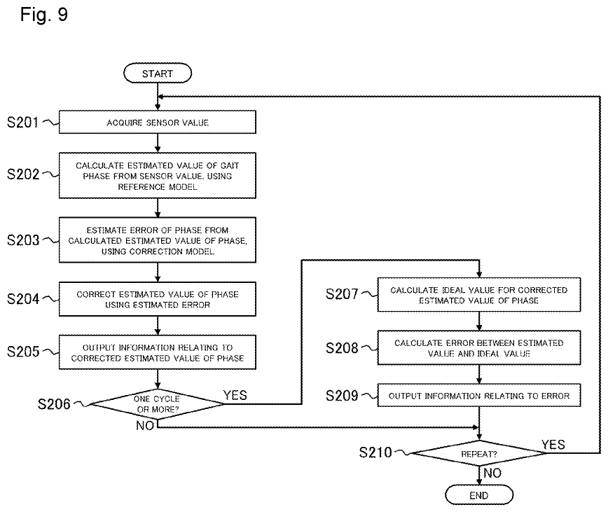

The phase estimation program 82 is a program for causing the phase estimation device 2 to execute information processing relating to estimation of the gait phase (see FIG. 9, which will be described later). The phase estimation program 82 includes a series of commands for the information processing. At least one of the phase estimation program 82, the reference model data 121, and the correction model data 125 may also be stored in the storage medium 92. The phase estimation device 2 may acquire at least one of the phase estimation program 82, the reference model data 121, and the correction model data 125 from the storage medium 92.

The phase estimation device 2 may perform data communication with another computer via the communication interface 23. The phase estimation device 2 may be connected via the communication interface 23 or the external interface 24 to the sensor S for obtaining the sensor value 51 during the estimation phase. When performing an intervention such as gait assistance based on the obtained estimated value 57 of the phase as output processing, the phase estimation device 2 may be connected to an intervention device (controlled device) such as a gait assistance device via the communication interface 23 or the external interface 24. An operator can operate the phase estimation device 2 by using the input device 25 and the output device 26. The input device 25 and the output device 26 may also be formed in one piece by, for example, a touch panel display or the like.

Note that, regarding the specific hardware configuration of the phase estimation device 2, constituent elements may be omitted, replaced, or added as appropriate according to the embodiment. For example, the controller 21 may also include a plurality of hardware processors. The type of the hardware processors is not particularly limited and may be selected as appropriate according to the embodiment. The storage 22 may also be constituted by a RAM and a ROM included in the controller 21. At least one of the communication interface 23, the external interface 24, the input device 25, the output device 26, and the drive 27 may also be omitted. The phase estimation device 2 may also be constituted by a plurality of computers. In this case, the hardware configurations of the computers may or may not match. Also, the phase estimation device 2 may be an information processing device designed exclusively for the service to be provided, or may be a general-purpose server device, a general-purpose PC, a tablet PC, a mobile terminal, or the like.

### [Software Configuration]

### <Model Generation Device>

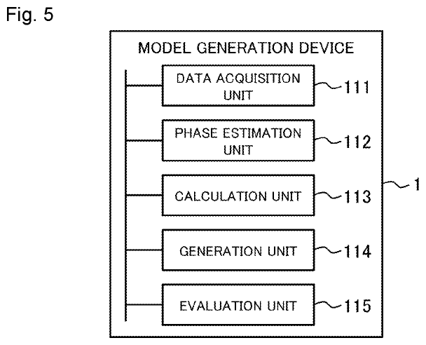

FIG. 5 schematically shows an example of the software configuration of the model generation device 1 according to the present embodiment. The controller 11 of the model generation device 1 expands the model generation program 81 stored in the storage 12 to the RAM. Then, the controller 11 causes the CPU to execute a command included in the model generation program 81 expanded to the RAM. As a result, the model generation device 1 according to this embodiment operates as a computer including a data acquisition unit 111, a phase estimation unit 112, a calculation unit 113, a generation unit 114, and an evaluation unit 115 as software modules. That is, in this embodiment, each software module of the model generation device 1 is realized by the controller 11 (CPU).

The data acquisition unit 111 is configured to acquire sensor data 31 generated by measuring walking of one or more cycles of the user Z, using the sensor S. The phase estimation unit 112 is configured to use the reference model 40 to calculate an estimated value 33 of the gait phase in the acquired sensor data 31. The calculation unit 113 is configured to calculate an ideal value 35 of the gait phase with respect to the calculated estimated value 33, based on the cycle of walking that appears in the sensor data 31. The generation unit 114 is configured to generate the correction model 45 by modeling the error between the estimated value 33 and the ideal value 35 of the gait phase.

In this embodiment, the sensor data 31 may also be generated by measuring walking of a plurality of cycles. After the phase estimation unit 112 calculates the estimated value 33 of the phase, the evaluation unit 115 is configured to calculate the variation of the calculated estimated value 33 of the phase, and if the magnitude of the variation of the estimated value 33 exceeds a threshold value, perform notification of an alert. Note that in this embodiment, the model generation device 1 may be configured to repeatedly execute a generation cycle including processing of the data acquisition unit 111, the phase estimation unit 112, the calculation unit 113, and the generation unit 114. When the generation cycle is repeatedly executed, the processing of the evaluation unit 115 may also be executed in each generation cycle.

### <Phase Estimation Device>

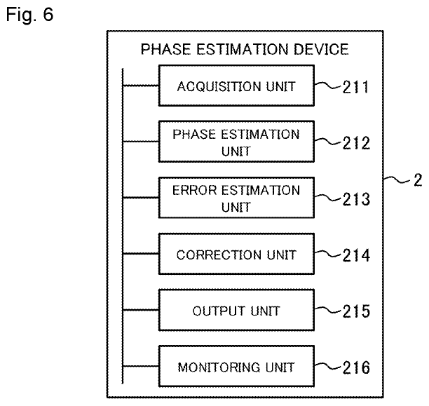

FIG. 6 schematically shows an example of the software configuration of the phase estimation device 2 according to this embodiment. The controller 21 of the phase estimation device 2 expands the phase estimation program 82 stored in the storage 22 to the RAM. Then, the controller 21 causes the CPU to execute a command included in the phase estimation program 82 expanded to the RAM. As a result, the phase estimation device 2 according to this embodiment operates as a computer including the acquisition unit 211, the phase estimation unit 212, the error estimation unit 213, the correction unit 214, the output unit 215, and the monitoring unit 216 as software modules. That is, in this embodiment, each software module of the phase estimation device 2 is also realized by the controller 21 (CPU).

The acquisition unit 211 is configured to acquire the sensor value 51 of the sensor S regarding the walking of the user Z. The phase estimation unit 212 is configured to use the reference model 40 to calculate an estimated value 53 of the gait phase from the acquired sensor value 51. The error estimation unit 213 is configured to use the correction model 45 to estimate the error 55 from the calculated estimated value 53 of the phase. The correction unit 214 is configured to correct the calculated estimated value 53 of the phase using the estimated error 55. Through the processing of the correction unit 214, the corrected estimated value 57 of the phase is obtained. The output unit 215 is configured to output information relating to the corrected estimated value 57 of the phase.

In the present embodiment, the phase estimation device 2 may be configured to repeatedly execute an estimation cycle including processing performed by the acquisition unit 211, the phase estimation unit 212, the error estimation unit 213, the correction unit 214, and the output unit 215. The monitoring unit 216 is configured to, in response to the estimation cycle being repeatedly executed for the walking of one or more cycles of the user Z, calculate an ideal value of the gait phase for the corrected estimated value 57 based on the cycle of walking, calculate an error between the corrected estimated value 57 and the ideal value, and output information relating to the calculated error.

### <Others>

The software modules of the model generation device 1 and the phase estimation device 2 will be described in detail in an operation example that will be described later. Note that this embodiment describes an example in which the software modules of the model generation device 1 and the phase estimation device 2 are both implemented by general-purpose CPUs. However, some or all of the above-described software modules may also be implemented by one or more dedicated processors. The above-described modules may also be realized as hardware modules. Also, regarding the software configurations of the model generation device 1 and the phase estimation device 2, software modules may be omitted, replaced, or added as appropriate according to the embodiment.

### § 3 Operation Example

### [Model Generation Device]

FIG. 7 is a flowchart showing an example of a processing procedure of the model generation device 1 according to the present embodiment. The processing procedure of the model generation device 1 described below is an example of a model generation method (information processing method). However, the processing procedure of the model generation device 1 described below is merely an example, and each step may be changed to the extent possible. Also, steps may be omitted, replaced, or added as appropriate in the following processing procedure according to the embodiment.

### (Step S101)

In step S101, the controller 11 operates as the data acquisition unit 111 and acquires the sensor data 31 generated by measuring walking of one or more cycles of the user Z, using the sensor S.

The user Z may be caused to perform a walking motion as appropriate (e.g., according to instructions from a physical therapist). The sensor S may perform measurements multiple times for walking of one cycle of the user Z. Accordingly, the sensor data 31 may be configured to include a plurality of sensor values. In this embodiment, the sensor data 31 may be generated by measuring walking of a plurality of cycles with the sensor S. The amount of the sensor data 31 (number of sensor values) and the sampling interval may be determined as appropriate according to the embodiment.

The sensor data 31 may be acquired at any timing when the walking of the user Z is observed. For example, the sensor data 31 may be acquired by measuring the walking of the user Z with the sensor S in order to generate the correction model 45. Also, for example, the sensor data 31 may be acquired while the phase estimation device 2 is performing estimation processing. In this case, at least some of the sensor data 31 may be constituted by the sensor values 51 acquired while repeatedly performing the estimation processing.

The acquired sensor data 31 may be stored in any storage region. The storage region may be, for example, a RAM of a controller (11, 21), the storage (12, 22), a storage medium (91, 92), an external storage device, another computer, or the like. The controller 11 may acquire at least some of the sensor data 31 from any storage region. Also, the controller 11 may directly acquire the sensor data 31 from at least the sensor S.

The status of the user Z when acquiring the sensor data 31 may be determined as appropriate according to the embodiment. In one example, the sensor data 31 may be generated by measuring the walking of the user Z while receiving gait assistance. In another example, the sensor data 31 may be generated by measuring the walking of the user Z while the user Z receives another intervention. In yet another example, the sensor data 31 may be generated by measuring the walking of the user Z while the user Z is not receiving any intervention (e.g., without any gait assistance).

Also, the elapsed time may be reflected in the acquisition of the sensor data 31. In one example, the controller 11 may acquire the sensor data 31 within a predetermined period from the time (current time) at which the processing of step S101 is executed. In another example, the controller 11 may add a weight corresponding to the elapsed time to the sensor data 31. Upon acquiring the sensor data 31, the controller 11 advances the processing to the next step S102.

### (Step S102)

In step S102, the controller 11 operates as the phase estimation unit 112, and calculates the estimated value 33 of the gait phase in the acquired sensor data 31 using the reference model 40.

In one example, the controller 11 may input the sensor values at each sampling time included in the sensor data 31 to the reference model 40, and execute calculation processing of the reference model 40. The calculation content of the reference model 40 may be determined as appropriate according to the embodiment. As a result of the execution of the calculation processing of the reference model 40, the controller 11 may acquire the estimated value 33 for the sensor value at each sampling time. Upon acquiring the estimated value 33, the controller 11 advances the processing to the next step S103.

Note that the controller 11 may initially set the reference model 40 to a usable state in the model generation device 1 by referring to the reference model data 121 at any timing before the processing of step S102 is executed. The reference model data 121 may be configured as appropriate such that the reference model 40 can be reproduced.

### (Step S103)

In step S103, the controller 11 operates as the calculation unit 113, and calculates the ideal value 35 of the gait phase with respect to the calculated estimated value 33 based on the cycle of walking that appears in the sensor data 31.

The controller 11 may calculate the ideal value 35 (true value) of the phase at each sampling time as appropriate by analyzing the cycle of walking that appears in the sensor data 31. In one example, the controller 11 may calculate the ideal value 35 corresponding to each estimated value 33 by equally dividing the phase with respect to the time from one heel strike to the next heel strike that appears in the sensor data 31. In another example, the controller 11 may calculate the ideal value 35 corresponding to each estimated value 33 by equally dividing the phase with respect to the time when the estimated value 33 obtained by the reference model 40 is 0 to 2π. Upon calculating the ideal value 35 corresponding to each estimated value 33, the controller 11 advances the processing to the next step S104.

### (Step S104)

In step S104, the controller 11 operates as the evaluation unit 115 and calculates the variation in the estimated value 33 of the phase calculated in each cycle of walking. The variation of the estimated value 33 may be expressed by known statistical values such as variance and standard deviation.

The controller 11 calculates the variation in the estimated value 33 for the same or an approximate phase in each cycle of walking. As a result, the controller 11 evaluates the variation in the estimated value 33 of the phase during each cycle of walking. In one example, the controller 11 may calculate the error between the estimated value 33 and the ideal value 35 of the phase that correspond to each other. Then, the controller 11 may use the ideal value 35 as a reference to calculate the variation in the error as the variation in the estimated value 33. However, the method for calculating the variation in the estimated value 33 is not limited to such an example, and may be determined as appropriate according to the embodiment. The processing of step S104 may be executed at any timing after the processing of step S102 is executed. Upon calculating the variation in the estimated value 33, the controller 11 advances the processing to the next step S105.

### (Step S105)

In step S105, the controller 11 operates as the evaluation unit 115 and determines whether or not the magnitude of the variation in the calculated estimated value 33 exceeds a threshold. The controller 11 determines the branch destination of the processing according to the result of the determination. If the magnitude of the variation of the estimated value 33 exceeds the threshold, the controller 11 advances the processing to step S106. On the other hand, if the magnitude of the variation in the estimated value 33 is less than the threshold, the controller 11 omits the processing of step S106 and step S107, and advances the processing to step S108. If the magnitude of the variation in the estimated value 33 is equal to the threshold value, the branch destination of the process may be either step S106 or step S108. The threshold may be provided through any method such as designation by the operator or a set value within a program.

### (Step S106 and Step S107)

In step S106, the controller 11 operates as the evaluation unit 115 and performs notification of an alert. In a typical example, the controller 11 may output an alert via the output device 16. However, the alert notification method need not be limited to this example, and may be determined as appropriate according to the embodiment. After performing notification of the alert, the controller 11 advances the processing to the next step S107.

In step S107, the controller 11 inquires of the operator via the output device 16 whether or not to generate the correction model 45. The controller 11 receives a response from the operator via the input device 15, and determines the branch destination of the processing according to the obtained response. When a response indicating that the correction model 45 is to be generated is obtained, the controller 11 advances the processing to step S108. On the other hand, when a response indicating that the correction model 45 is not to be generated is obtained, the controller 11 ends the processing procedure of the model generation device 1 according to this operation example.

### (Step S108)

In step S108, the controller 11 operates as the generation unit 114 and generates the correction model 45 by modeling the error between the estimated value 33 and the ideal value 35 of the gait phase.

The correction model 45 may be configured as appropriate to be able to calculate an error corresponding to the estimated value of the gait phase from the estimated value. In one example, the controller 11 may calculate the error between the estimated value 33 and the ideal value 35 of the phase that correspond to each other. In the processing of step S104, if an error has been calculated, the processing may be omitted. When the sensor data 31 includes sensor values for walking of a plurality of cycles, the controller 11 may calculate the average value of the error with respect to the estimated value among the cycles. If a weight corresponding to the elapsed time has been added, the controller 11 may calculate the average value of the errors in the cycles using a weighted average. As a result, the controller 11 may calculate an error with respect to the estimated value of the gait phase. The controller 11 may generate the correction model 45 by modeling the calculated error.

FIG. 8 shows an example of the correction model 45 according to this embodiment. In one example, the controller 11 may generate, as the correction model 45, a function expression that expresses an error with respect to the estimated value (for example, a function expression that expresses the graph in FIG. 8) using a method such as fitting or machine learning. In another example, the controller 11 may generate the correction model 45 by plotting errors with respect to estimated values to create a data table of the plotted errors. Upon generating the correction model 45, the controller 11 advances the processing to the next step S109. Note that the data in FIG. 8 was obtained under the same conditions as a later-described experimental example.

### (Step S109)

Returning to FIG. 7, in step S109, the controller 11 operates as the generation unit 114 and generates information relating to the generated correction model 45 as the correction model data 125. The correction model data 125 may be configured as appropriate such that the correction model 45 can be reproduced. The controller 11 stores the generated correction model data 125 in a predetermined storage region.

The predetermined storage region may be selected as appropriate. The predetermined storage region may be, for example, the RAM of the controller 11, the storage 12, the storage medium 91, an external storage device, or the like. The external storage device may be, for example, a data server, an externally-attached storage device, or the like. When the storage of the correction model data 125 is complete, the controller 11 advances the processing to the next step S110.

Note that the generated correction model data 125 may be provided to the phase estimation device 2 using any method and at any timing. In one example, the controller 11 may transfer the correction model data 125 to the phase estimation device 2 as the processing of step S109 or separately from the processing of step S109. The phase estimation device 2 may acquire the correction model data 125 (correction model 45) by receiving this transfer. In another example, the phase estimation device 2 may obtain the correction model data 125 (correction model 45) by using the communication interface 23 to access the model generation device 1 or the data server. In yet another example, the phase estimation device 2 may acquire the correction model data 125 (correction model 45) via the storage medium 92.

### (Step S110)

In step S110, the controller 11 determines whether or not to repeat the generation cycle including steps S101 to S103 and step S108.

The criteria for determination may be set as appropriate according to the embodiment. In one example, the number of instances of repeating generation of the correction model 45 may be set using a threshold. The threshold may be provided by any method such as designation by the operator or a set value within a program. In this case, the controller 11 may count the number of instances of repeating the processing of steps S101 to S109. When the counted number of instances of repetition is less than a threshold, the controller 11 may determine that execution of the generation cycle is to be repeated. On the other hand, when the number of instances of repetition reaches the threshold, the controller 11 may determine that execution of the generation cycle is not to be repeated (execution of the generation cycle is to be ended).

In another example, the controller 11 may inquire to the operator whether or not to repeatedly execute the generation cycle. The controller 11 may receive a response from the operator via the input device 15 and determine whether or not to repeatedly execute the generation cycle based on the obtained response. As a result, the model generation device 1 may be configured to execute a generation cycle and then execute the next generation cycle in response to a request from an operator.

If it is determined that execution of the generation cycle is to be repeated, the controller 11 returns to step S101 and executes the processing again starting from step S101.

When the generation cycle is to be repeatedly executed, the controller 11 may acquire new sensor data 31 in the current generation cycle through the processing of step S101. When new sensor data 31 is acquired, the controller 11 may store the acquired new sensor data 31 in an any storage region. The stored sensor data 31 may be used to generate the correction model 45 in subsequent generation cycles.

Also, when the generation cycle is to be repeatedly executed, in one example, in the second generation cycle and onward, the controller 11 may correct the reference model 40 using the correction model 45 generated in the previous generation cycle. As a result, the controller 11 may generate the corrected reference model 40 (i.e., may update the reference model 40). In this case, in the processing of step S102 in the second generation cycle and onward, the controller 11 may use the corrected reference model 40 to calculate the estimated value 33 of the gait phase in the sensor data 31 acquired in the current generation cycle. Then, in step S108, the controller 11 may generate a new correction model 45 with respect to the corrected reference model 40. Also, the controller 11 may update the reference model data 121 to indicate the corrected reference model 40, and provide the updated reference model data 121 to the phase estimation device 2 at any timing. As a result, the corrected reference model 40 may also be used in the estimation processing in the phase estimation device 2. In another example, the controller 11 may repeat the processing of the generation cycle and update the correction model 45 without updating the reference model 40.

Also, if the generation cycle is to be repeatedly executed, in one example, the controller 11 may use all of the sensor data 31 acquired up to the current generation cycle to generate the correction model 45. In another example, the controller 11 may use some of the sensor data 31 acquired up to the current generation cycle (e.g., sensor data for which the elapsed time since acquisition is less than a threshold) to generate the correction model 45. In another example, the controller 11 may also use only the new sensor data 31 acquired in the current generation cycle to generate the correction model 45. In another example, the controller 11 may also receive, from the operator, a designation of the sensor data 31 to be used to generate the correction model 45 in the current generation cycle. In this case, the controller 11 may generate the correction model 45 using the sensor data 31 specified by the operator.

On the other hand, if it is determined that the execution of the generation cycle is not to be repeated, the controller 11 ends the processing procedure of the model generation device 1 according to this operation example.

After completing the processing procedure according to this operation example, the controller 11 may execute the processing again starting from step S101 at any timing. In one example, the controller 11 may execute the processing again starting from step S101 in response to a request from the operator via the input device 15. As a result, similarly to step S110, the model generation device 1 may be configured to execute a generation cycle and then execute the next generation cycle in response to a request from the operator. Regarding executing the processing again starting from step S101, it may be the same as the case where the generation cycle is repeatedly executed in step S110.

Note that the model generation device 1 may generate the correction model 45 by executing the processing from step S101 at any timing. In one example, the model generation device 1 may generate the correction model 45 as pre-processing performed when starting observation of the walking of the user Z (e.g., starting assistance by a gait assistance device). In a situation where the gait assistance device performs assistance, the model generation device 1 may generate the correction model 45 according to an assistance pattern given to the user Z.

### <Phase Estimation Device>

FIG. 9 is a flowchart showing an example of a processing procedure of the phase estimation device 2 according to the present embodiment. The processing procedure of the phase estimation device 2 described below is an example of a phase estimation method (information processing method). However, the processing procedure of the phase estimation device 2 described below is merely an example, and each step may be changed to the extent possible. Also, regarding the following processing procedure, steps may be omitted, replaced, or added as appropriate according to the embodiment.

Note that, as pre-processing, the controller 21 may output instruction information for prompting the user Z to start a walking motion to the output device 26 or another output device of the computer. The user Z may perform the walking motion on a device such as a treadmill. The controller 21 may start executing the processing from step S201 in response to the user Z starting the walking motion.

### (Step S201)

In step S201, the controller 21 operates as the acquisition unit 211 and acquires the sensor value 51 of the sensor S regarding the walking of the user Z. In one example, the controller 21 may acquire the sensor value 51 directly from the sensor S. In another example, the controller 21 may obtain the sensor value 51 indirectly from the sensor S via another computer or the like. Upon acquiring the sensor value 51, the controller 21 advances the processing to the next step S202.

### (Step S202)

In step S202, the controller 21 operates as the phase estimation unit 212, and calculates the estimated value 53 of the gait phase from the acquired sensor value 51, using the reference model 40. If the corrected reference model 40 has been obtained by the model generation device 1, the controller 21 may use the corrected reference model 40 to calculate the estimated value 53 of the phase from the sensor value 51.

In one example, the controller 21 may input the acquired sensor value 51 to the reference model 40 and execute calculation processing of the reference model 40. As a result of this calculation processing, the controller 21 may acquire an estimated value 53 of the phase with respect to the sensor value 51. For example, when the reference model 40 is constituted by a function formula, the controller 21 may acquire the estimated value 53 of the phase by substituting the sensor value 51 into the function formula and executing calculation of the function formula. The function formula may be configured by a machine learning model such as a neural network. Also, for example, when the reference model 40 is configured as a data table, the controller 21 may extract the estimated value 53 of the phase corresponding to the sensor value 51 from the data table. Also, for example, when the reference model 40 is configured by a rule, the controller 21 may calculate the estimated value 53 of the phase by applying the rule to the sensor value 51. Upon acquiring the estimated value 53 of the phase, the controller 21 advances the processing to the next step S203. Note that the controller 21 may initially set the reference model 40 to a usable state in the phase estimation device 2 by referring to the reference model data 121 at any timing before the processing of step S202 is executed.

### (Step S203)

In step S203, the controller 21 operates as the error estimation unit 213, and uses the correction model 45 to estimate the error 55 from the calculated estimated value 53 of the phase.

In one example, the controller 21 may input the calculated estimated value 53 of the phase to the correction model 45 and execute the calculation processing of the correction model 45. The calculation content of the correction model 45 may be determined as appropriate according to the embodiment. In the example of FIG. 8, as the calculation processing of the correction model 45, the controller 21 may substitute the estimated value 53 of the phase into a function formula and execute the calculation processing of the function formula. In another example, when the correction model 45 is expressed as a data table, the controller 21 may extract the error 55 corresponding to the estimated value 53 of the phase from the data table as the calculation processing of the correction model 45. As a result of the calculation processing of the correction model 45, the controller 21 can acquire the error 55 that is estimated in correspondence with the estimated value 53 of the phase. Upon acquiring the estimated error 55, the controller 21 advances the processing to the next step S204. Note that the controller 21 may initially set the correction model 45 to a usable state in the phase estimation device 2 by referring to the correction model data 125 at any timing before the processing of step S203 is executed.

### (Step S204)

In step S204, the controller 21 operates as the correction unit 214 and corrects the calculated estimated value 53 of the phase using the estimated error 55. As a result, the controller 21 acquires the corrected estimated value 57 of the phase.

The content of the correction processing may be determined as appropriate according to the expression format of the error 55. In one typical example, the error 55 may be expressed in the form of a sum or a difference. In response to this, the controller 21 may acquire the corrected estimated value 57 of the phase by calculating the sum or difference of the estimated value 53 of the phase and the error 55. Upon acquiring the corrected estimated value 57 of the phase, the controller 21 advances the processing to the next step S205.

### (Step S205)

In step S205, the controller 21 operates as the output unit 215 and outputs information relating to the corrected estimated value 57 of the phase.

The output destination and the content of the information to be output may be determined as appropriate according to the embodiment. For example, the controller 21 may output the corrected estimated value 57 of the phase as-is to the output device 26 or an output device of another computer. In one example, the controller 21 may display a graph on the display and plot the corrected estimated value 57 of the phase on the graph. Also, for example, the controller 21 may perform some sort of information processing based on the obtained estimated value 57. The controller 21 may output the result of the information processing as information relating to the estimated value 57 of the phase. Outputting the result of this information processing may include controlling the operation of the controlled device according to the estimated value 57 of the phase. The controlled device may be, for example, an intervention device such as a gait assistance device, an electrical stimulation device, or an activation measurement device. In one example, the controller 21 may control a gait assistance operation performed by the gait assistance device by determining an assistance amount from the corrected estimated value 57 of the phase according to a set assistance pattern, and outputting the determined assistance amount to the gait assistance device. In this embodiment, the controller 21 may execute at least one of these output processes as the processing of step S205. When the output of the information is complete, the controller 21 advances the processing to the next step S206.

### (Step S206)

In step S206, the controller 21 determines whether or not the walking of the user Z has been measured for one cycle or more (i.e., whether or not the sensor value 51 for one cycle or more has been obtained).

In this embodiment, the phase estimation device 2 is configured to be able to repeatedly execute an estimation cycle including step S201 to step S205 through the processing of step S210, which will be described later. In view of this, in one example, the controller 21 may determine that the walking of the user Z has been measured for one cycle or more in response to the estimated value 57 obtained in each estimation cycle moving from one cycle to the next cycle (in one example, changing again from 0 after exceeding 2π) by repeatedly executing the estimation cycle for walking of one or more cycles. When the walking of the user Z is measured for one cycle or more, the controller 21 advances the processing to the next step S207.

On the other hand, the controller 21 may determine that measurement of one cycle or more has not yet been performed in response to the estimated value 57 obtained in each estimation cycle staying within the range of 0 to 2π of a certain cycle. If measurement of one cycle or more has not been performed, the controller 21 omits the processing of steps S207 to S209 and advances the processing to step S210.

### (Step S207 to Step S209)

In step S207, the controller 21 operates as the monitoring unit 216, and calculates an ideal value of the gait phase with respect to the corrected estimated value 57, based on the cycle of walking. The method for calculating the ideal value may be the same as the processing of step S103. Upon calculating the ideal value corresponding to the estimated value 57, the controller 21 advances the processing to the next step S208.

In step S208, the controller 21 operates as the monitoring unit 216 and calculates the error between the estimated value 57 of the phase and the ideal value that correspond to each other. After calculating the error of the estimated value 57 obtained in each estimation cycle, the controller 21 advances the processing to the next step S209.

In step S209, the controller 21 operates as the monitoring unit 216 and outputs information relating to the calculated error.

The output destination and the content of the information to be output may be determined as appropriate according to the embodiment. For example, the controller 21 may output the calculated error as-is to the output device 26 or an output device of another computer. As an example, the controller 21 may display a graph on the display and plot the error of the estimated value 57 on the graph, based on the ideal value.

Also, for example, the controller 21 may determine whether or not the calculated error exceeds a threshold. The threshold may be provided in any manner. If the calculated error exceeds the threshold, the controller 21 may output an alert to the output device 26 or an output device of another computer as information relating to the error.

The operator may determine whether or not the correction model 45 used for the estimation processing no longer matches the walking motion of the user Z, based on at least one of the error and the alert that are output. Then, if it is determined that the correction model 45 no longer matches the user Z, the operator may send a request to the model generation device 1 to generate a new correction model 45, via the input device 15 or a computer other than the model generation device 1. In response to this, the model generation device 1 (controller 11) may generate a new correction model 45 by executing the processing from step S101. At this time, at least some of the plurality of sensor values 51 acquired while repeating the estimation cycle may be used as the sensor data 31 to generate the new correction model 45. Also, the model generation device 1 (controller 11) may collect new sensor data 31 in order to generate a new correction model 45.

Also, for example, if the calculated error exceeds a threshold, the controller 21 may output an instruction to the model generation device 1 to prompt generation of a new correction model 45. In response to this, the model generation device 1 may execute the processing from step S101.

In this embodiment, the controller 21 may execute at least one of these output processes as the processing of step S209. After outputting the information relating to the error, the controller 21 advances the processing to the next step S210.

Note that the controller 21 may execute the processing of steps S207 to S209 at any timing after the sensor S measures the walking of one or more cycles of the user Z. In one example, the controller 21 may execute the processing of step S207 to step S209 each time one cycle of the measured walking elapses. In another example, the controller 21 may execute the processing of steps S207 to S209 once on the measurement results of a plurality of gait cycles.

### (Step S210)

In step S210, the controller 21 determines whether or not to repeatedly execute the estimation cycle including steps S201 to S205.

The criteria for determination may be set as appropriate according to the embodiment. In one example, the controller 21 may determine whether or not to repeatedly execute the estimation cycle based on an index such as the number of repetitions, time, or number of instances of walking. In this case, the threshold for the index may be provided through any method. When the calculated index is less than the threshold, the controller 21 may determine that execution of the estimation cycle is to be repeated. On the other hand, when the calculated index reaches the threshold, the controller 21 may determine that execution of the estimation cycle is not to be repeated (execution of the estimation cycle is to be ended).

In another example, the controller 21 may determine that the estimation cycle is to be repeated until an end instruction is given from the operator. The end instruction may be given via the input device 25. Then, after receiving an end instruction from the operator, the controller 21 may determine that execution of the estimation cycle is not to be repeated (execution of the estimation cycle is to be ended).

If it is determined that the estimation cycle is to be repeated, the controller 21 returns to step S201 and executes the processing again from step S201. As a result, the controller 21 continuously estimates the gait phase of the user Z. On the other hand, if it is determined that the estimation cycle is not to be repeated, the controller 21 ends the processing procedure of the phase estimation device 2 according to the present operation example.

After ending the processing procedure according to this operation example, the controller 21 may execute the processing again from step S201 at any timing (i.e., the controller 21 may resume the execution of the processing from step S201). In one example, the controller 21 may execute the processing again from step S201 in response to a request from the operator given via the input device 25. Regarding executing the processing again from step S201, it may be the same as the case where the estimation cycle is repeatedly executed in step S210.

While repeatedly executing the estimation cycle, the plurality of sensor values 51 acquired through the processing of step S201 may be stored as the sensor data 31 in any storage area. The model generation device 1 may generate the correction model 45 using the sensor data 31 including the plurality of sensor values 51 obtained by the phase estimation device 2. The generated correction model 45 (correction model data 125) may be provided to the phase estimation device 2 at any timing.

### [Characteristics]

As described above, in the processing of step S108, generating the correction model 45 is merely using the sensor data 31 to model the error between the estimation result (estimated value 33) of the reference model 40 and the true value (ideal value 35). In the above-described processing of step S103, it is easy to calculate the ideal value 35 (true value) of the gait phase based on the cycle of walking that appears in the sensor data 31. For this reason, the correction model 45 can be easily created for each user Z. Also, it is easy to generate the correction model 45 again according to circumstances such as the situation of the user Z, such that the gait phase can be estimated with high accuracy. Furthermore, the generated correction model 45 is merely configured to show the correspondence between the estimated value of the reference model 40 and the error (that is, calculate the error from the estimated value). For this reason, calculation of the correction model 45 in the processing of step S203 is easy.

Accordingly, with the model generation device 1 according to the present embodiment, by correcting the output (estimated value) of the reference model 40, the correction model 45 that makes it possible to easily and accurately estimate the gait phase in real time can be generated. According to the phase estimation device 2 according to the present embodiment, by using such a correction model 45, the gait phase of the user Z can be estimated easily and accurately in real time.

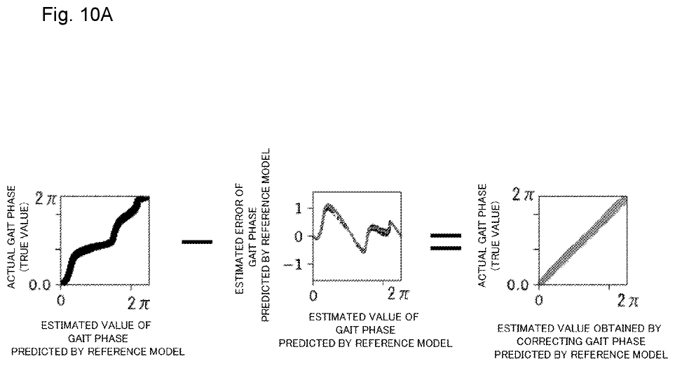

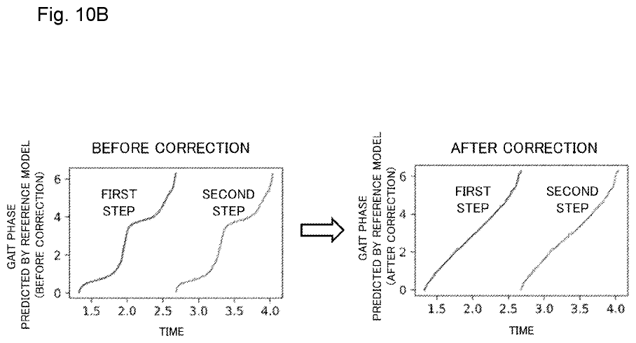

FIG. 10A schematically shows an example of a process of processing for calculating a corrected estimated value 57 of the phase using the correction model 45 according to the present embodiment in the processing of steps S202 to S204. FIG. 10B shows an example of estimated values (53, 57) of the phase obtained before and after correction by the correction model 45. While repeating the estimation cycle, the phase estimation device 2 (controller 21) uses the correction model 45 in the processing of step S203 to calculate the error 55 for the estimated value 53 obtained in step S202 described above. Then, the phase estimation device 2 (controller 21) acquires the corrected estimated value 57 of the phase by correcting the estimated value 53 using the obtained error 55 in the above-described processing of step S204. As described above, the model generation device 1 can easily generate the correction model 45 that matches the walking motion of the user Z. Due to the correction model 45 being appropriately generated, as illustrated in FIGS. 10A and 10B, in the processing of step S204, the estimated value 57 that changes linearly in the range of 0 to 2π (i.e., the estimated value 57 with little distortion relative to the true value) can be obtained in real time. Note that the data in FIGS. 10A and 10B were obtained under the same conditions as the experimental examples described later. Also, the data in FIG. 10B was obtained by plotting, in time series, the estimated values (53, 57) of the phase that were calculated in real time at 250 Hz. Note that in the examples of FIGS. 10A and 10B, the gait phase is expressed as a value of 0 to 2π, but the expression format of the phase need not be limited to such examples. The gait phase may be output in another expression format. In another example, the gait phase (0 to 2π) may also be expressed as 0% to 100%.

Also, in the present embodiment, when generating the correction model 45, variation in the estimated value 33 of the phase can be monitored through the processing of steps S104 to S106. If the estimated value 33 of the phase has a large amount of variation, the error between the estimated value 33 and the ideal value 35 will also vary, and therefore there is a possibility that the accuracy of the generated correction model 45 will deteriorate. According to the present embodiment, variation in the estimated value 33 of the phase can be monitored, and if the variation is large, an alert can be issued to make it visible that such a possibility exists. As a result, through the processing of step S107, it is possible to stop the generation of the correction model 45 or to avoid continued use of a correction model 45 with poor accuracy in the phase estimation device 2.

Also, in the present embodiment, while the phase estimation device 2 repeatedly executes the estimation cycle, the accuracy of the correction performed by the correction model 45 that is being used can be evaluated through the processing of steps S206 to S209. There is a possibility that the walking motion of the user Z will change over time. When the walking motion of the user Z changes and the correction model 45 no longer matches the walking motion of the user Z, as shown in FIGS. 10A and 10B, it becomes difficult to accurately estimate the gait phase of the user Z (to obtain a linear estimated value 57). According to this embodiment, by evaluating the accuracy of the correction performed by the correction model 45, it is possible to make visible whether or not the correction model 45 being used is in such a state. This makes it possible to avoid continued use of a correction model 45 that no longer matches the walking motion of the user Z for phase estimation. Also, by prompting the generation of a new correction model 45, it is possible to maintain the accuracy of phase estimation in the phase estimation device 2.

Also, in this embodiment, the generation cycle may be repeatedly executed in the model generation device 1. When repeating the generation cycle, a corrected reference model 40 may be generated by correcting the reference model 40 using the correction model 45 generated in the previous generation cycle. As a result, it can be expected that the accuracy of phase estimation by the reference model 40 and the correction model 45 will improve.

### § 4 Modified Examples

Although an embodiment of the present invention has been described in detail above, the above description is merely an illustration of the present invention in all respects. In the embodiment described above, various improvements or changes may be made as appropriate. For example, the following changes are possible. Note that hereinafter, the same reference signs are used for components that are the same as in the above-described embodiment, and description of points that are the same as in the above-described embodiment is omitted as appropriate. The following modified examples can be combined as appropriate.

<4.1>

The estimation system according to the above-described embodiment may be applied to any situation in which the gait phase of a person is estimated. Situations in which the gait phase of a person is estimated may be a situation in which a gait assistance device assists the walking of a person, a situation in which spasticity is induced through functional electrical stimulation, a situation in which the activation of spinal nerve pathways during walking are measured, and a situation in which an abnormality in walking is detected. The estimation result of the gait phase (estimated value 57) may be used, for example, to determine the assistance amount, determine the timing of applying electrical stimulation, detect an abnormality in walking, and the like. Specific examples with limited application situations are shown below.

(A) Situation where Operation of Controlled Device is Controlled

As an example, the control device may be configured to execute: a step of acquiring the sensor value 51 of the sensor S with respect to walking of the user Z; a step of calculating an estimated value 53 of the gait phase from the acquired sensor value 51 using the reference model 40; a step of estimating the error 55 from the calculated estimated value 53 of the phase, using the correction model 45; a step of correcting the calculated estimated value 53 of the phase using the estimated error 55; a step of determining a drive amount of the controlled device from the corrected estimated value 57 of the phase; and a step of outputting the determined drive amount. The controlled device may be a gait assistance device, an electrical stimulation device, or an activation measurement device. Determining the drive amount of the controlled device may be determining the assistance amount of the gait assistance device, determining the amount of electrical stimulation by the electrical stimulation device, or determining the amount of electrical stimulation in the activation measurement device. Determining an amount may include determining whether or not to perform provision thereof. Outputting the determined drive amount means controlling the operation of the controlled device (driving the controlled device) according to the determined drive amount, or indirectly controlling the operation of the controlled device by providing the determined drive amount to a controller of the controlled device.

(A-1) Situation where Gait Assistance is Performed

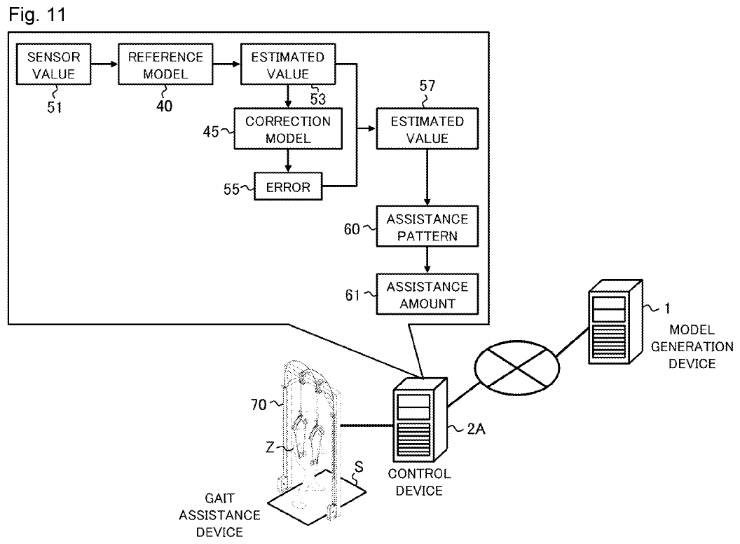

FIG. 11 schematically shows an example of an application situation of the estimation system according to a first specific example. The first specific example is an example in which the above embodiment is applied to a situation where the estimation result of the gait phase is utilized for gait assistance. The estimation system according to the first specific example includes a model generation device 1 and a control device 2A. The control device 2A is an example of the phase estimation device 2 described above. A gait assistance device 70 is an example of a controlled device.

In the first specific example, the control device 2A sets an assistance pattern 60. The control device 2A acquires the sensor value 51 of the sensor S regarding the walking of the user Z. The control device 2A uses the reference model 40 to calculate the estimated value 53 of the gait phase from the acquired sensor value 51. The control device 2A uses the correction model 45 to estimate the error 55 from the calculated estimated value 53 of the phase. The control device 2A corrects the calculated estimated value 53 of the phase, using the estimated error 55. As a result, the control device 2A acquires the corrected estimated value 57 of the phase. The control device 2A determines the assistance amount 61 of the gait assistance device 70 from the corrected estimated value 57 of the phase according to the set assistance pattern 60. The control device 2A outputs the determined assistance amount 61 in order to control the gait assistance device 70. That is, in the first specific example, outputting the information relating to the estimated value 57 of the phase includes determining the assistance amount 61 from the corrected estimated value 57 of the phase in accordance with the set assistance pattern 60, and outputting the determined assistance amount 61. When repeatedly executing the estimation cycle, the control device 2A may also monitor whether or not an amount of change in the corrected estimated value 57 satisfies a permissible condition. Except for these points, the configuration of the first specific example may be the same as that of the above-described embodiment.

### [Hardware Configuration]

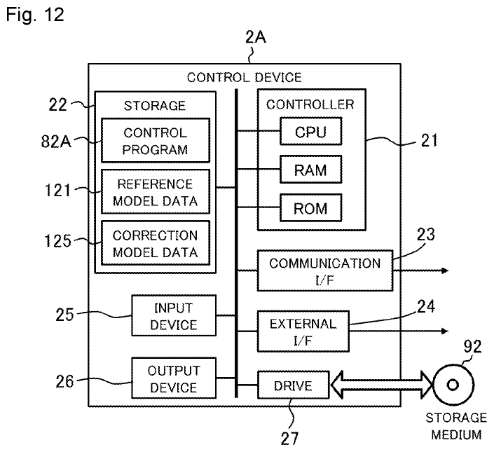

FIG. 12 schematically shows an example of a hardware configuration of the control device 2A according to the first specific example. As shown in FIG. 12, the hardware configuration of the control device 2A may be similar to the above-described hardware configuration of the phase estimation device 2.

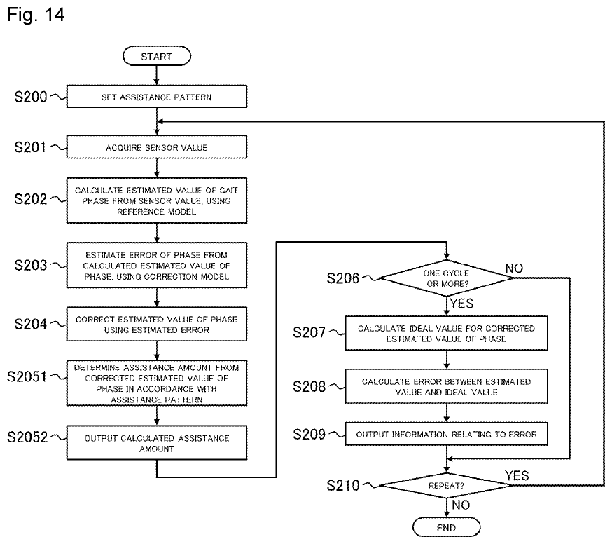

In the example of FIG. 12, the storage 22 stores a control program 82A. The control program 82A is a program for causing the control device 2A to execute information processing (FIG. 14, which will be described later) relating to estimation of the gait phase and control of the gait assistance device 70. The control program 82A includes a series of instructions for the information processing. The control program 82A is an example of the phase estimation program 82. The control program 82A may also be stored in the storage medium 92. The control device 2A may also acquire the control program 82A from the storage medium 92.

Also, the control device 2A may be connected to the gait assistance device 70 via the communication interface 23 or the external interface 24. The gait assistance device 70 is configured to provide assistance through intervention (e.g., force, electrical stimulation, etc.) to the user Z performing a walking motion. The configuration of the gait assistance device 70 is not particularly limited as long as it can provide assistance for walking, and may be determined as appropriate according to the embodiment. A known gait assistance device may be used as the gait assistance device 70. In one example, a body-weight-relieving device proposed in the reference document (WO 2020/246587) may be used as the gait assistance device 70. In another example, an assistance device configured to provide an assistance force to an ankle joint as illustrated in FIGS. 12 to 14 of the reference document (WO 2017/138651) may be used as the gait assistance device 70. In addition, for example, an assistance device configured to provide an assistance force to the knee joint and the ankle joint, as exemplified in Japanese Patent Application Publication No. 2010-264019, Japanese Patent Application Publication No. 2017-213246, and the like may be used as the gait assistance device 70.

### [Software Configuration]

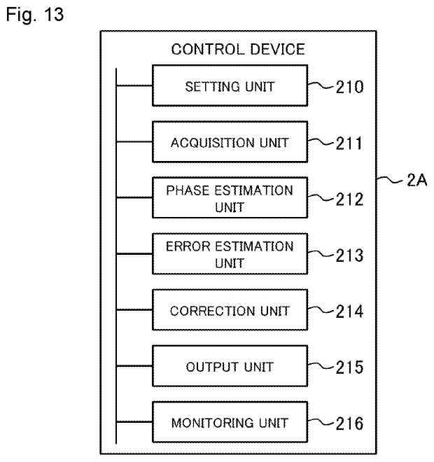

FIG. 13 schematically shows an example of the software configuration of the control device 2A according to the first specific example. Similarly to the above-described embodiment, the controller 21 of the control device 2A executes the control program 82A. As a result, the control device 2A operates as a computer including each software module.

In the example of FIG. 13, the control device 2A further includes a setting unit 210. The setting unit 210 is configured to set the assistance pattern 60. Also, in the first specific example, the output unit 215 is configured to determine the assistance amount 61 from the corrected estimated value 57 of the phase according to the set assistance pattern 60, and output the determined assistance amount 61.

Furthermore, in the first specific example, the control device 2A may be configured to repeatedly execute an estimation cycle including the processing of the acquisition unit 211, the phase estimation unit 212, the error estimation unit 213, the correction unit 214, and the output unit 215. In the step of determining the assistance amount 61 in the second estimation cycle and onward, the output unit 215 may be further configured to detect the amount of change between the corrected estimated value 57 in the previous estimation cycle and the corrected estimated value 57 in the current estimation cycle, and determine whether or not the calculated amount of change satisfies a permissible condition. Then, if the amount of change satisfies the permissible condition, the output unit 215 may be configured to determine the assistance amount 61 in the current estimation cycle from the corrected estimated value 57 in the current estimation cycle, and if the amount of change does not satisfy the permissible condition, the output unit 215 determines the assistance amount 61 in the current estimation cycle based on the corrected estimated value 57 in the previous estimation cycle, regardless of the corrected estimated value 57 in the current estimation cycle.

Note that, similarly to the above-described embodiment, some or all of the software modules of the control device 2A may be realized by one or more dedicated processors. Also, regarding the software configuration of the control device 2A, software modules may be omitted, replaced, or added as appropriate according to the embodiment.

### Operation Example

FIG. 14 is a flowchart showing an example of a processing procedure of the control device 2A according to the first specific example. The processing procedure of the control device 2A described below is an example of a control method (information processing device) of the gait assistance device 70. However, the processing procedure of the control device 2A described below is merely an example, and each step may be changed to the extent possible. Also, regarding the following processing procedure, steps may be omitted, replaced, or added as appropriate according to the embodiment.

In the example of FIG. 14, the processing procedure of the control device 2A further includes the processing of step S200 before step S201. Also, the above step S205 is composed of step S2051 and step S2052. Except for these points, the processing procedure of the control device 2A may be the same as the processing procedure of the phase estimation device 2 described above. The processing of other steps (S201 to S204, S206 to S210) may be the same as in the above embodiment.

### (Step S200)

In step S200, the controller 21 operates as the setting unit 210 and sets the assistance pattern 60. The assistance pattern 60 defines the assistance amount for the gait phase. The assistance pattern 60 may be given in advance. In this case, the setting information of the assistance pattern 60 may be held as, for example, a predetermined storage region (the storage 22, etc.), a setting value within a program, or the like. The setting information may be provided system-specifically, or may be provided through prior input by the operator. The controller 21 may also set the assistance pattern 60 based on this setting information. Alternatively, the assistance pattern 60 may be provided through input from an operator. In one example, the assistance pattern 60 may be generated through manual input by an operator (physical therapist). However, manually generating the assistance pattern 60 depends on the experience of a skilled person and is difficult. In view of this, in another example, the assistance pattern 60 may be constituted by one or more muscle modules.

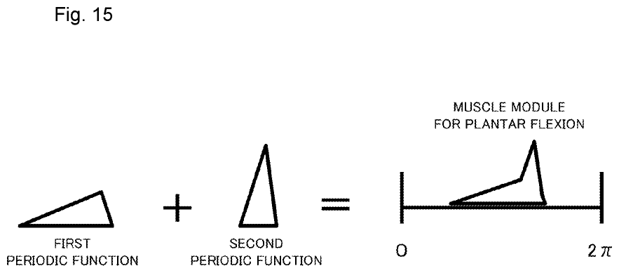

FIG. 15 schematically shows an example of muscle modules that constitute the assistance pattern 60 according to the first specific example. A muscle module may be constituted by combining a plurality of periodic functions to reproduce muscle synergy such as knee flexion, knee extension, plantar flexion, foot dorsiflexion, anti-gravity muscles, and the like, for example. Muscle synergy is the coordinated activity of a plurality of muscles. Muscle synergy may be non-linear. When reproducing muscle synergy, for example, a periodic function with low calculation cost, such as a rectangular wave or a sawtooth wave, may be preferentially used.

In FIG. 15, an example of a muscle module for plantar flexion is shown. In the example of FIG. 15, the muscle module for plantar flexion is constituted by a combination of two sawtooth waves (first periodic function, second periodic function) of different sizes. Thus, by appropriately selecting the type and size of the periodic function, nonlinear muscle synergy can be reproduced relatively easily. According to the configuration of this muscle module, it is possible to realize the assistance pattern 60 in accordance with muscle synergy through easy calculation. Also, as a conventional method, there is a method of defining a position (e.g., a target position of a motor) with respect to a gait phase in an assistance pattern. In this method, when a plurality of assistance patterns are combined, the intention of each assistance pattern is lost. Therefore, it is not easy to combine a plurality of assistance patterns. On the other hand, since muscle modules define forces (e.g., torque, pressure) for the gait phase, even if a plurality of muscle modules are combined, the intention of each muscle module is not impaired. For this reason, a plurality of muscle modules can be easily combined. In addition, by selecting one or more muscle modules and designating the strength of the selected muscle module, it is possible to easily create an assistance pattern 60 that is appropriate for muscle synergy without relying on the experience of a skilled person. That is, compared to the case where the assistance pattern 60 is generated manually, there are fewer parameters to be adjusted (muscle module selection, strength designation, etc.), and therefore the amount of labor required to generate the assistance pattern 60 can be reduced. Note that the assistance pattern 60 may also include a muscle module that suppresses muscle activity. Such a muscle module may be configured as appropriate by reproducing muscle synergy that suppresses muscle activity (e.g., subtracting).

Note that the assistance pattern 60 constituted by this muscle module may be independently adopted in any form that does not use the correction model 45. That is, the configuration of the muscle module may be employed in all situations in which assistance patterns are set. In one example, the computer may set an assistance pattern, calculate an estimated value of the gait phase of the user using a predetermined method, determine an assistance amount from the calculated estimated value in accordance with the set assistance pattern, and output the assistance amount. The predetermined method for estimating the gait phase may be selected as appropriate according to the embodiment. A known method may be adopted as the predetermined method. In this case, the assistance pattern may be constituted by one or more muscle modules, and the muscle module may be constituted by a combination of a plurality of periodic functions so as to reproduce muscle synergy.

### (Step S2051 and Step S2052)

Returning to FIG. 14, in step S2051, the controller 21 operates as the output unit 215 and determines the assistance amount 61 from the corrected estimated value 57 of the phase in accordance with the set assistance pattern 60. That is, the controller 21 refers to the assistance pattern 60 and specifies the assistance amount 61 for the corrected estimated value 57 of the phase (acquires information indicating the assistance amount 61 from the assistance pattern 60). Upon determining the assistance amount 61, the controller 21 advances the processing to the next step S2052.

In step S2052, the controller 21 operates as the output unit 215 and outputs the determined assistance amount 61 in order to control the gait assistance device 70. In one example, when the control device 2A is directly connected to the gait assistance device 70, outputting the determined assistance amount 61 may be driving the gait assistance device 70 with the determined assistance amount 61. In another example, when the gait assistance device 70 includes a control device, outputting the determined assistance amount 61 may be transmitting a drive command including information indicating the determined assistance amount 61 to the control device, and causing the control device to drive the gait assistance device 70 with the determined assistance amount 61. Upon outputting the assistance amount 61, the controller 21 advances the processing to the next step S206.

### (Others)

According to the determination result in step S210, the controller 21 repeatedly executes an estimation cycle including steps S201 to S204, step S2051, and step S2051.

While repeatedly executing the estimation cycle, the estimated value 57 of the phase calculated from the sensor value 51 shows unexpected behavior such as changing rapidly or changing in a retrograde manner in some cases due to the user Z performing a walking motion that is different from the expected walking motion (in one example, the foot getting caught on the ground). In order to deal with this, in the first specific example, in step S2051 of the second estimation cycle and onward, the controller 21 may calculate the amount of change between the corrected estimated value 57 in the previous estimation cycle and the corrected estimated value 57 in the current estimation cycle. Then, the controller 21 may determine whether or not the calculated amount of change satisfies the permissible condition.

The permissible condition may be set as appropriate so as not to allow unexpected behavior of the estimated value 57. In one example, a permissible range (for example, 0 to an upper limit) of the amount of change may be defined as the permissible condition. The threshold of the upper limit may be provided through any method. As a result, if the amount of change in the estimated value 57 is negative or exceeds the threshold (upper limit) within the same gait cycle, it may be determined that the permissible condition is not satisfied. Note that when the range of the phase is defined as 0 to 2π, the estimated value 57 of the phase can vary from a value close to 2π to a value close to 0 when moving from one gait cycle to the next. The permissible condition may be set such that the fluctuation in the estimated value 57 due to normal walking is not determined as a sudden change or a retrograde change.

If the amount of change in the estimated value 57 satisfies the permissible condition, in the processing of step S2051, the controller 21 may determine the assistance amount 61 in the current estimation cycle from the corrected estimated value 57 in the current estimation cycle. On the other hand, if the amount of change does not satisfy the permissible condition, the controller 21 may determine the assistance amount 61 in the current estimation cycle based on the corrected estimated value 57 in the previous estimation cycle, regardless of the corrected estimated value 57 in the current estimation cycle. The corrected estimated value 57 of the phase in the current estimation cycle may be discarded.

In one example, determining the assistance amount 61 based on the previous estimated value 57 may be using the previous assistance amount 61 as the current assistance amount 61. That is, the controller 21 may adopt the assistance amount 61 in the previous estimation cycle as-is as the assistance amount 61 in the current estimation cycle. If the amount of change does not satisfy the permissible condition, the controller 21 may control the assistance of the walking by assuming that there is no change in the phase.

In another example, determining the assistance amount 61 based on the previous estimated value 57 may be acquiring the current assistance amount 61 by correcting the previous assistance amount 61 with consideration given to the amount of change accompanying the elapse of time for one cycle. For example, the controller 21 may further correct the corrected estimated value 57 from the cycle of walking or step width based on the amount of time for one cycle. Then, the controller 21 may determine the assistance amount 61 in the current estimation cycle from the further-corrected estimated value in accordance with the assistance pattern 60. Alternatively, the controller 21 may calculate the amount of change corresponding to the amount of time for one cycle in accordance with the assistance pattern 60. Then, the controller 21 may calculate the assistance amount 61 in the current estimation cycle by correcting the assistance amount 61 in the previous estimation cycle using the calculated amount of change.

Note that, similarly to the above-described embodiment, the plurality of sensor values 51 obtained through the processing of step S201 may be stored as the sensor data 31 while the estimation cycle is repeatedly executed. In one example, the sensor data 31 may be generated by using the sensor S to measure walking while receiving assistance from the gait assistance device 70. If the correction model 45 has already been generated, the control device 2A may control the assistance performed by the gait assistance device 70 through the above-described processing of steps S201 to S2052. While executing this control, the sensor data 31 (sensor value 51) of walking while receiving assistance may be acquired. On the other hand, if the correction model 45 has not been generated, the control device 2A may omit the processing of steps S203 and S204, determine the assistance amount from the estimated value 53 of the phase obtained by the reference model 40, and control the assistance of the gait assistance device 70 with the determined assistance amount. While executing this control, the sensor data 31 (sensor value 51) of walking while receiving assistance may be acquired. In another example, the sensor data 31 may be generated by using the sensor S to measure walking without receiving assistance from the gait assistance device 70.

Also, the model generation device 1 may repeatedly generate the correction model 45 (i.e., may repeatedly execute the generation cycle). In one example, the model generation device 1 may correct the reference model 40 using the correction model 45 generated in the previous generation cycle. Accordingly, the control device 2A may use the corrected reference model 40 in the processing of step S202. In another example, the model generation device 1 may repeat the generation cycle processing and update the correction model 45 without updating the reference model 40. In either mode, if the correction model 45 has already been generated, the control device 2A may use the correction model 45 to control the assistance performed by the gait assistance device 70, as described above. While this control is being executed, new sensor data 31 (sensor value 51) may be acquired, and the acquired new sensor data 31 may be used to generate the correction model 45 in the subsequent generation cycles. In yet another example, the model generation device 1 may generate the correction model 45 by executing generation cycle processing in both a situation where the gait assistance device 70 is assisting walking and a situation where the gait assistance device 70 is not performing assistance. The control device 2A may switch the correction model 45 that is to be used in a situation where the gait assistance device 70 performs walking assistance and a situation where assistance is omitted.

### (Characteristics)

With the control device 2A according to the first specific example, similarly to the above-described embodiment, the gait phase of the user Z can be estimated easily and accurately in real time by using the correction model 45. As a result, walking assistance can be performed for the user Z at an appropriate timing.

Also, in the first specific example, the user Z may be a patient with hemiplegia or the like, and the assistance provided by the gait assistance device 70 may be utilized as at least a part of rehabilitation. In this case, by repeatedly receiving walking assistance from the gait assistance device 70, the walking ability of the user Z may improve and the walking of the user Z may change. If the walking of the user Z changes, the correction model 45 may no longer match the walking motion of user Z, and it may become difficult to accurately estimate the phase of walking of the user Z. On the other hand, according to the first specific example, by evaluating the accuracy of the correction performed by the correction model 45 through the processing of steps S206 to S209, it is possible to visualize whether or not the correction model 45 that is being used is in such a state. This makes it possible to avoid continued use of the correction model 45 that no longer matches the walking motion of the user Z for phase estimation. Also, by prompting generation of a new correction model 45, it is possible to maintain the accuracy of phase estimation in the control device 2A and continue to perform walking assistance at an appropriate timing.

Also, according to the first specific example, while the estimation cycle is repeatedly executed, it is possible to monitor whether or not the amount of change of the estimated value 57 of the phase satisfies the permissible condition in the processing of step S2051. As a result, even if the estimated value 57 of the phase exhibits unexpected behavior, it is possible to perform appropriate assistance using the estimation result of the previous estimation cycle.

Note that in the above-described first specific example, the gait assistance device 70 may be configured to assist walking using the output of pneumatic artificial muscles. As an example of the gait assistance device 70 configured in this manner, the body-weight-relieving device proposed in the above-mentioned reference document (WO 2020/246587) may be used. An example of the assistance pattern 60 illustrated in FIG. 15 is not smoothed and therefore has excellent visibility for the operator (physical therapist), but if the drive amount is output with the shape of this assistance pattern 60 as-is, it is difficult to provide smooth assistance. In contrast, by using a gait assistance device 70 that is configured to assist walking using the output of a pneumatic artificial muscle, the assistance amount actually provided by the gait assistance device 70 is smoothed due to the dynamics of the pneumatic artificial muscles (e.g., due to delays in the motor driver, etc.) even if the assistance pattern 60 is not smoothed. For this reason, according to this configuration, it is not necessary to smooth the assistance pattern 60, and therefore smooth assistance can be expected without increasing the amount of calculation. Also, the visibility of the assistance pattern 60 can be ensured (e.g., it is easy to specify the start and end points of the assistance). Note that the control device 2A may also predict the assistance amount to be output by the gait assistance device 70 by applying the assistance pattern 60 to the dynamics of the pneumatic artificial muscle. The control device 2A may also output the predicted assistance amount to an output device. As a result, the assistance amount that will actually be output may be presented to the operator (physical therapist). However, the configuration of the first specific example need not be limited to such an example. In another example, the controller 21 of the control device 2A may smooth the set assistance pattern 60, and may also determine the assistance amount 61 according to the smoothed assistance pattern 60. Smoothing may be performed using any method.

(A-2) Situation where Spasticity is Attenuated Through Functional Electrical Stimulation

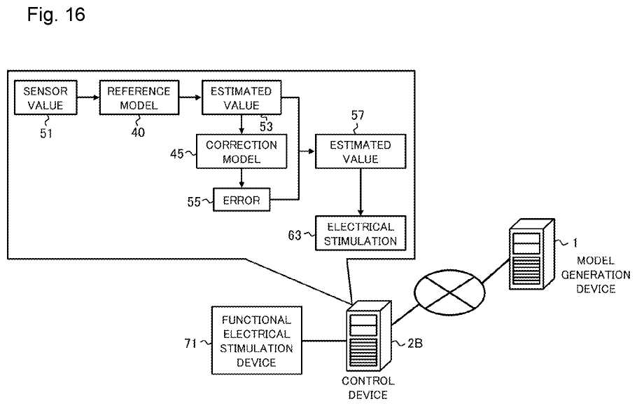

FIG. 16 schematically shows an example of an application situation of the estimation system according to a second specific example. The second specific example is an example in which the above-described embodiment is applied to a situation where the estimation result of the phase of walking is utilized to determine the timing of providing functional electrical stimulation. The estimation system according to the second specific example includes a model generation device 1 and a control device 2B. The control device 2B is an example of the above-described phase estimation device 2. The functional electrical stimulation device 71 is an example of a controlled device (electrical stimulation device).

Spasticity is a condition in which muscles become so tense that it becomes difficult to move the limbs or they move on their own. Spasticity appears as an after-effect of stroke in some cases. There are reports stating that muscle spasticity is attenuated and static standing balance and walking speed are improved by performing training using functional electrical stimulation on a patient with this type of spasticity. The control device 2B according to the second specific example is configured to control the operation of the functional electrical stimulation device 71 and provide this training to the user Z.

Specifically, the control device 2B acquires the sensor value 51 of the sensor S regarding the walking of the user Z. The control device 2B uses the reference model 40 to calculate the estimated value 53 of the gait phase from the acquired sensor value 51. The control device 2B uses the correction model 45 to estimate the error 55 from the calculated estimated value 53 of the phase. The control device 2B corrects the calculated estimated value 53 of the phase using the estimated error 55. As a result, the control device 2B acquires the corrected estimated value 57 of the phase.

The control device 2B determines whether or not to provide functional electrical stimulation 63 according to the corrected estimated value 57 of the phase. Determining whether or not to provide the functional electrical stimulation 63 is an example of determining the amount of electrical stimulation provided by the electrical stimulation device. The timing of providing the functional electrical stimulation 63 may be defined according to the gait phase, as in the assistance pattern 60 of the above-described first specific example. The definition information of this timing may be stored in any storage region such as a storage of the control device 2B. The control device 2B may determine whether or not to provide the functional electrical stimulation 63 according to the corrected estimated value 57 of the phase by referring to the definition information.

Then, the control device 2B outputs information indicating the result of the determination in order to control the functional electrical stimulation device 71. In one example, if the control device 2B is directly connected to a functional electrical stimulation device 71, the control device 2B may drive the functional electrical stimulation device 71 so as to provide the functional electrical stimulation 63 to the user Z in response to the control device 2B determining that the functional electrical stimulation 63 is to be provided. In another example, if the functional electrical stimulation device 71 includes a control device, the control device 2B may, in response to determining that the functional electrical stimulation 63 is to be provided, transmit a drive command including information indicating the determination to the control device, and drive the functional electrical stimulation device 71 so as to provide the functional electrical stimulation 63 to the user Z.

That is, in the second specific example, outputting the information relating to the estimated value 57 of the phase includes determining whether or not to provide the functional electrical stimulation 63 according to the corrected estimated value 57 of the phase, and outputting information indicating the result of determination. Except for these points, the configuration of the second specific example may be the same as that of the above-described embodiment. The hardware configuration and software configuration of the control device 2B may be the same as those of the above-described phase estimation device 2 or the control device 2A. In the second specific example, the phase estimation program may be read as a control program, as in the first specific example. Also, the processing procedure of the control device 2B may be the same as that of the above-described phase estimation device 2 or the control device 2A.

Note that the control device 2B may be connected to the functional electrical stimulation device 71 via a communication interface or an external interface. The functional electrical stimulation device 71 is configured to provide functional electrical stimulation to the user Z performing a walking motion. Functional electrical stimulation refers to electrical stimulation that is configured to achieve a specific function (e.g., simulate neural activity). The configuration of the functional electrical stimulation device 71 need not be particularly limited as long as it can provide functional electrical stimulation, and may be determined as appropriate according to the embodiment. As the functional electrical stimulation device 71, a known functional electrical stimulation device may be used.

### (Characteristics)

With the control device 2B according to the second specific example, similarly to the above-described embodiment, by using the correction model 45, it is possible to accurately estimate the gait phase of the user Z easily and in real time. As a result, the functional electrical stimulation 63 can be provided to the user Z at appropriate timing. As a result, appropriate training can be provided to the user Z, and an improvement in static standing balance and walking speed can be expected.

(A-3) Situation where Activation of Spinal Nerve Pathways is Measured while Walking

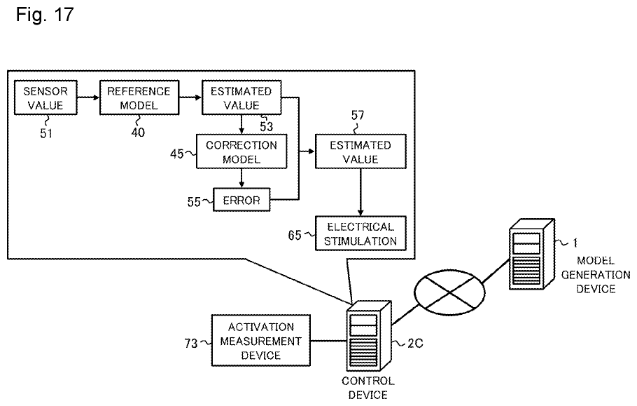

FIG. 17 schematically shows an example of an application situation of the estimation system according to a third specific example. When measuring the activation of spinal nerve pathways, electrical stimulation may be provided according to the gait phase. The third specific example is an example in which the above-described embodiment is applied to a situation where the estimation result of the gait phase is utilized to determine the timing of providing this electrical stimulation. The estimation system according to the third specific example includes the model generation device 1 and a control device 2C. The control device 2C is an example of the above-described phase estimation device 2. The activation measurement device 73 is an example of a controlled device.

In the third specific example, the control device 2C acquires the sensor value 51 of the sensor S regarding the walking of the user Z. The control device 2C uses the reference model 40 to calculate the estimated value 53 of the gait phase from the acquired sensor value 51. The control device 2C uses the correction model 45 to estimate the error 55 from the calculated estimated value 53 of the phase. The control device 2C corrects the calculated estimated value 53 of the phase using the estimated error 55. As a result, the control device 2C obtains the corrected estimated value 57 of the phase.

The control device 2C determines whether or not to provide the electrical stimulation 65 according to the corrected estimated value 57 of the phase. Determining whether or not to provide the electrical stimulation 65 is an example of determining the amount of electrical stimulation in the activation measurement device. The timing of providing the electrical stimulation 65 may be defined according to the gait phase, as in the above-described second specific example. The definition information of this timing may be stored in any storage region such as the storage of the control device 2C. The control device 2C may determine whether or not to provide the electrical stimulation 65 according to the corrected estimated value 57 of the phase by referring to the definition information.

Then, the control device 2C outputs information indicating the determination result in order to control the activation measurement device 73. The activation measurement device 73 may include an electrical stimulation device and a myoelectricity measurement device. The electrical stimulation device may be configured to provide electrical stimulation. A known electrical stimulation device may be used as the electrical stimulation device. The myoelectricity measurement device may be configured to measure reflections (myoelectricity such as H-waves and M-waves) caused by electrical stimulation. A known myoelectricity measurement device may be used as the myoelectricity measurement device as well.

In one example, if the control device 2C is directly connected to the activation measurement device 73, the control device 2C may measure the myoelectricity of the user Z with the myoelectricity measurement device and drive the electrical stimulation device so as to provide the electrical stimulation 65 to the user Z in response to determining that the electrical stimulation 65 is to be provided. In another example, if the activation measurement device 73 includes a control device, the control device 2C may instruct the control device to cause the myoelectricity measurement device to measure the myoelectricity of the user Z, and in response to determining that the electrical stimulation 65 is to be provided, transmit a drive command including information indicating the determination to the control device to cause the control device to drive the electric stimulation device so as to provide the electrical stimulation 65 to the user Z.

That is, in the third specific example, outputting the information relating to the estimated value 57 of the phase includes determining whether or not to provide the electrical stimulation 65 according to the corrected estimated value 57 of the phase, and outputting information indicating the determination result. Except for these points, the configuration of the third specific example may be the same as that of the above-described embodiment. The hardware configuration and the software configuration of the control device 2C may be the same as those of the phase estimation device 2 or the control device 2A. In the third specific example, the phase estimation program may be read as a control program, as in the above-described first specific example. Also, the processing procedure of the control device 2C may be the same as that of the phase estimation device 2 or the control device 2A. Note that the control device 2C may be connected to the activation measurement device 73 via a communication interface or an external interface.

### (Characteristics)

With the control device 2C according to the third specific example, similarly to the above-described embodiment, by using the correction model 45, it is possible to easily and accurately estimate the gait phase of the user Z in real time. As a result, the electrical stimulation 65 can be provided to the user Z at an appropriate timing. As a result, activation of the spinal nerve pathway can be appropriately measured in order to evaluate the degree of inhibition or facilitation of the spinal nerve pathway.

(B) Situation where Abnormality in Walking is Detected

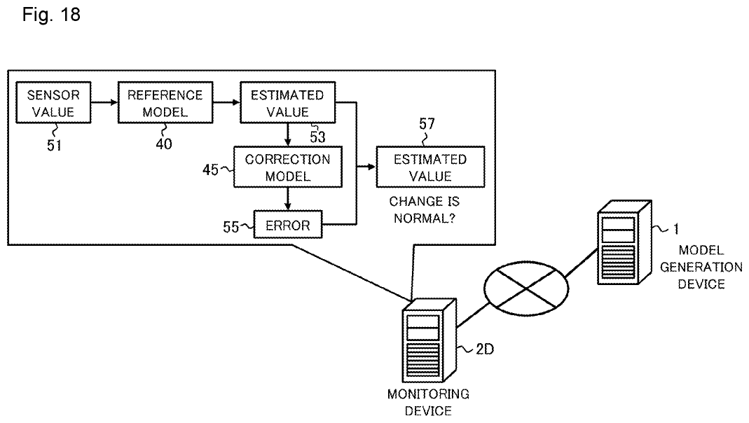

FIG. 18 schematically shows an example of an application situation of the estimation system according to a fourth specific example. The fourth specific example is an example in which the above-described embodiment is applied to a situation where an abnormality in walking is detected on the basis of the estimation result of the gait phase. The estimation system according to the fourth specific example includes a model generation device 1 and a monitoring device 2D. The monitoring device 2D is an example of the above-described phase estimation device 2.

In the fourth specific example, the monitoring device 2D acquires the sensor value 51 of the sensor S regarding the walking of the user Z. The monitoring device 2D uses the reference model 40 to calculate the estimated value 53 of the gait phase from the acquired sensor value 51. The monitoring device 2D uses the correction model 45 to estimate the error 55 from the calculated estimated value 53 of the phase. The monitoring device 2D corrects the calculated estimated value 53 of the phase using the estimated error 55. As a result, the monitoring device 2D obtains the corrected estimated value 57 of the phase.

The monitoring device 2D determines whether or not there is an abnormality in the walking of the user Z based on the corrected estimated value 57 of the phase. While the user Z is walking normally, the obtained estimated value 57 of the phase changes linearly, as shown in FIG. 10A above. On the other hand, if an abnormality occurs in the walking of the user z, the obtained estimated value 57 of the phase deviates from the normal pattern (straight line). The situation where an abnormality occurs in walking is, for example, a situation where there is a risk of falling. The monitoring device 2D may determine whether or not there is an abnormality in the walking of the user Z according to deviation from this normal pattern. The information indicating the normal pattern may be held as definition information, similarly to the above-described second specific example.

In one example, the monitoring device 2D may determine whether or not there is an abnormality in the walking of the user Z by calculating the magnitude of the deviation between the estimated value 57 of the phase and the normal pattern, and comparing the calculated magnitude of the deviation with a threshold. In another example, the monitoring device 2D may use a calculation model such as a trained machine learning model to evaluate the deviation of the estimated value 57 of the phase from a normal pattern, and determine whether or not there is an abnormality in the walking according to the evaluation result.

Then, the monitoring device 2D outputs information indicating the determination result. In one example, outputting the information indicating the determination result may be outputting the determination result to the monitoring device 2D or an output device of another computer. In another example, outputting information indicating the determination result may be, if it is determined that there is an abnormality in the walking of the user Z, outputting an alert for performing notification of that fact to the monitoring device 2D or the output device of another computer. In yet another example, outputting information indicating the determination result may be, if it is determined that there is an abnormality in the walking of the user Z, controlling the operation of the gait assistance device so as to assist the walking of the user Z (e.g., so as to prevent falling).

As an example of a control method, when the monitoring device 2D is directly connected to a gait assistance device, the monitoring device 2D may drive the gait assistance device so as to assist the walking of the user Z in response to determining that there is an abnormality in the walking of the user Z. As another example, when the walking assistance device includes a control device, in response to determining that there is an abnormality in the walking of user Z, the monitoring device 2D may transmit a drive command including information indicating the determination to the control device to cause the control device to drive the gait assistance device so as to assist the walking of the user Z. The gait assistance device may be the same as the gait assistance device 70 described above.

That is, in the fourth specific example, outputting the information relating to the estimated value 57 of the phase includes determining whether or not there is an abnormality in the walking of the user Z based on the corrected estimated value 57 of the phase, and outputting information indicating the determination result. Except for these points, the configuration of the fourth specific example may be the same as in the above-described embodiment. The hardware configuration and the software configuration of the monitoring device 2D may be the same as those of the above-described phase estimation device 2 or the control device 2A. In the fourth specific example, the phase estimation program may be read as a monitoring program. Also, the processing procedure of the monitoring device 2D may be the same as that of the above-described phase estimation device 2 or the control device 2A. Note that the monitoring device 2D may be connected to the gait assistance device via a communication interface or an external interface.

### (Characteristics)

With the monitoring device 2D according to the fourth specific example, similarly to the above-described embodiment, by using the correction model 45, the gait phase of the user Z can be estimated easily and accurately in real time. As a result, it is possible to accurately estimate in real time whether or not there is an abnormality in the walking of the user Z. As a result, it is possible to perform an intervention (e.g., assistance to prevent falling) with respect to the walking of the user Z at an appropriate timing.

<4.2>

Regarding the processing procedures (FIGS. 7, 9, and 14) of the above-described embodiments and modified examples, at least one of omission, replacement, and addition of steps may be performed.

For example, in the processing procedure of the model generation device 1, the processing of steps S104 to S107 may be omitted. Accordingly, the evaluation unit 115 may be omitted from the software configuration of the model generation device 1. In the processing procedure of the model generation device 1 described above, the processing of step S107 may be omitted. In this case, the controller 11 may advance the processing to step S108 after the processing of step S106.

In the above-described processing procedure of the model generation device 1, the processing of step S106 may be omitted. If the magnitude of the variation in the estimated value 33 exceeds a threshold, the controller 11 may exclude the data of the cycle that is the cause of the large variation (typically, the data at the beginning of walking/just before the end of walking) to suppress the magnitude of variation, and then generate the correction model 45. In one example, the controller 11 may specify outliers among the estimated values 33 in each cycle, exclude the outliers, and then generate the correction model 45. A known statistical method may be employed to specify outliers.

In the above-described processing procedure of the model generation device 1 described above, the processing of step S110 may be omitted. If the model generation device 1 and the phase estimation device 2 are formed in one piece, the processing of step S109 may be omitted in the above-described processing procedure of the model generation device 1.

Also, for example, an important section may be provided in the cycle of walking (range of 0 to 2π). The important section may be provided through any method such as designation by an operator or a set value in a program. In the above-described step S108, the controller 11 may increase the weight of error evaluation for the important section relative to other sections, and thereby adjust the parameters of the correction model 45 such that the accuracy of correction in the important section is higher than that in other sections. As a result, it is possible to improve the accuracy of estimating the gait phase in the important section.

Also, for example, in the above-described processing procedure of the phase estimation device 2 (control device 2A), the processing of steps S206 to S209 may be omitted. Accordingly, the monitoring unit 216 may be omitted from the software configuration of the phase estimation device 2 (control device 2A). In the above-described processing procedure of the phase estimation device 2 (control device 2A), the processing of step S210 may be omitted. In another example, the controller 21 may repeatedly execute the estimation cycle until a stop instruction is given by an interrupt.

In the above-described processing procedure of the phase estimation device 2 (control device 2A), if the error calculated through the processing in step S208 exceeds the threshold, the controller 21 may output a notification performing indicating that fact to the model generation device 1. In response to receiving the notification, the model generation device 1 may execute processing from step S101 to generate a new correction model 45. The model generation device 1 may provide the new correction model 45 to the phase estimation device 2 (control device 2A) using any method. The phase estimation device 2 (control device 2A) may resume execution of the estimation cycle in response to receiving the new correction model 45. If the model generation device 1 and the phase estimation device 2 (control device 2A) are formed in one piece, the computer may also operate as the model generation device 1 to generate a new correction model 45 in response to the error calculated through the processing of step S208 exceeding a threshold.

Also, for example, at a stage where the correction model 45 has not been generated, the controller 21 may omit the processing of step S203 and step S204. In this case, the controller 21 may use the estimated value 53 of the phase obtained from the reference model 40 through the processing of step S202 to execute the output processing of step S205. In the above-described step S2051, the controller 21 may determine the assistance amount 61 from the estimated value 53 of the phase.

Also, in the estimation stage of the above-described embodiment, there is a possibility that a delay will occur (i.e., the phase of a slightly delayed time is estimated) between when the sensor value 51 is obtained and when the estimated value 53 of the phase is obtained due to the influence of the transmission of the sensor value 51 (e.g., a delay in data communication), the calculation load of the estimation processing including pre-processing (e.g., filtering) for obtaining the sensor value 51, and the like. As an example, if a configuration is adopted in which a plantar sensor (sensor S) is placed in an insole and data is transmitted via wireless communication, there is a possibility that a delay will occur between when the sensor value 51 is obtained and the estimated value 53 of the phase is obtained due to the influence of a delay in wireless communication. In addition, if a mode is adopted in which the controlled devices are driven (control devices 2A to 2C), there is a possibility that a delay will occur in the output process. As an example, if the controlled devices include an actuator, a delay may occur because it takes time from when a set value is (drive amount) is input to the actuator to when the output is actually obtained.

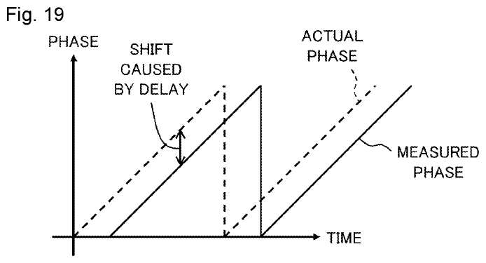

FIG. 19 is a diagram for describing a delay relating to phase estimation. In FIG. 19, the solid line indicates the measured phase (corrected estimated value 57 of the phase), and the dotted line indicates the actual phase at the time of reflection (e.g., the phase at the timing when walking is actually assisted by the gait assistance device 70). As mentioned above, the delay relating to phase estimation can occur in any one of the process of obtaining the sensor value 51 (transmission of the sensor value 51, etc.), the calculation process of obtaining the corrected estimated value 57 of the phase (pre-processing, the above-described estimation processing, etc.), and the process of outputting an estimation result (driving a controlled device, etc.). The effect of this delay can cause a discrepancy between the actual phase and the measured phase. In order to deal with this, the phase estimation device 2 (control devices 2A to 2C, monitoring device 2D) may be configured to specify the phase shift caused by the delay relating to phase estimation, and further correct the corrected estimated value 57 of the phase according to the specified shift. The amount of correction according on the delay may be estimated as appropriate based on the amount of change in the phase per unit time.

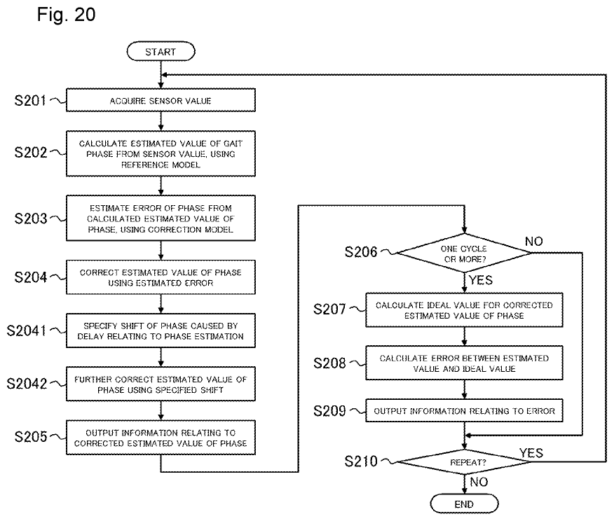

FIG. 20 is a flowchart showing another example of the processing procedure of the above-described phase estimation device 2. The processing procedure of the phase estimation device 2 described below is an example of a phase estimation method (information processing method). However, the processing procedure of the phase estimation device 2 described below is merely an example, and each step may be changed to the extent possible. Also, regarding the following processing procedure, steps may be omitted, replaced, or added as appropriate according to the embodiment.

In the example of FIG. 20, the processing procedure of the phase estimation device 2 further includes the processing of step S2041 and step S2042 between step S204 and step S205. Except for these points, the processing procedure of the phase estimation device 2 may be the same as the processing procedure of the phase estimation device 2 shown in FIG. 9 above. The processing of other steps (S201 to S204, S205 to S210) may be the same as in the above embodiment.

### (Step S2041)

In step S2041, the controller 21 operates as the correction unit 214 and specifies a phase shift caused by a delay relating to phase estimation. In one example, the controller 21 may calculate the phase shift caused by the delay by calculating “amount of change in phase per unit time×delay time”. Information on the amount of change in the phase per unit time and the delay time may be obtained as appropriate.

The amount of change in phase per unit time may be estimated from the angular velocity of the phase during walking. As an example of a simple method, in the process of repeating phase estimation, the controller 21 may calculate an estimated value of the amount of change in the phase per unit time by dividing the difference between the estimated value 57 in the previous instance of sampling and the estimated value 57 in the current instance sampling by the sampling period. The controller 21 may appropriately smooth the estimated value of the amount of change calculated at each sampling timing, and may use the smoothed estimated value as the “amount of change in phase per unit time” in the above calculation. Note that when this method is adopted, at the stage where a plurality of samples have not been obtained (at the time of initial calculation), the processing of step S2041 and step S2042 may be performed as appropriate (e.g., they may be omitted).

On the other hand, the delay time may be obtained in advance. Delays relating to phase estimation may include at least one of a delay in the process of acquiring the sensor value 51 (transmission of the sensor value 51, etc.), a delay in the calculation process (pre-processing, the above-described estimation processing, etc.) of calculating the corrected estimated value 57 of the phase, and a delay in the process of outputting the estimation result (driving the controlled device, etc.). The delay time may be measured using any method. As an example of a method for obtaining the delay time in the process of acquiring the sensor value 51, the delay time can be measured by performing measurement simultaneously by a sensor that is of the same type as the sensor S and whose transmission delay can be ignored, and the sensor S, and comparing the acquired data. As a specific example, when adopting a configuration in which sensor data of the sensor S is transmitted through wireless communication, the delay time may be obtained by connecting the sensor S (wireless sensor) and a wired sensor to a computer and calculating the delay in the sensor data obtained from the sensor S with respect to the sensor data obtained from the wired sensor on a computer. In another example, the delay time may also be obtained from known information about the transmission standard of the sensor S (e.g., in Bluetooth (registered trademark), the general delay time of each codec is known). In another example, the delay time may also be obtained by measuring the amount of time from when a pulse-shaped stimulus (e.g., a load) is applied to the sensor S under computer control, until when the sensor value for that stimulus is obtained. As an example of a method for obtaining the delay time in the calculation process, the amount of time required for the calculation from when the sensor value 51 is obtained until when the information is output may be measured, and the measured amount of time may be obtained as the delay time. The time required for the calculation may be specified as appropriate according to the calculation ability of the CPU of the computer (phase estimation device 2). As an example of a method for obtaining the delay time in the output process, an instruction may be provided to the controlled device and the amount of time required for output according to the instruction to be obtained from the controlled device may be obtained as the delay time. As a specific example, the controlled device may include an actuator (e.g., a case where, in the gait assistance device 70, the assistance amount is output by the actuator). In this case, the amount of time from when an instruction is given to the actuator under computer control until when output is actually obtained may be measured as the delay time. The point in time when output is actually obtained may be specified from the measured value of a sensor such as a load cell, a pressure sensor (if a fluid pressure actuator is used), or an ammeter (if a motor-driven actuator is used). Note that the delay time in the output process may vary dynamically. For example, in pneumatic artificial muscles, the delay time is short when increasing the internal pressure, but the delay time may be long when decreasing the internal pressure. For this reason, the delay time in the output process may also be configured to change according to the gait phase (estimated value 57). As an example, in a situation where the above-described gait assistance device 70 is controlled, the delay time in the output process may be determined according to the assistance pattern 60 and the estimated value 57. For example, the delay time in the output process may be constituted by a first delay time in the process of increasing the assistance amount and a second delay time in the process of decreasing the assistance amount. The control device 2A may distinguish whether the point in time of the assistance amount 61 determined from the estimated value 57 is in the increasing process or the decreasing process of the assistance amount in the assistance pattern 60, and select which of the first delay time or the second delay time is to be used. The value of each delay time may be determined according to the characteristics of the gait assistance device 70 (valve characteristics, etc.). Note that due to the difference between the first delay time and the second delay time, if the delay time to be used is switched at one point in time, the specified phase shift (correction amount) may become discontinuous. In order to deal with this, when switching from the first delay time to the second delay time, and when switching from the second delay time to the first delay time, the control device 2A may interpolate the value of the delay time between the first delay time and the second delay time, and asymptotically change the delay time to be used. The phase estimation device 2 may hold information on these delay times in advance. The controller 21 may calculate the phase shift caused by the delay by acquiring information on the amount of change in the phase per unit time and the delay time using any of the above methods, and calculating “amount of change in phase per unit time×delay time” according to the acquired information. Upon specifying the phase shift, the controller 21 advances the processing to the next step S2042.

### (Step S2042)

In step S2042, the controller 21 operates as the correction unit 214, and further corrects the corrected estimated value 57 of the phase based on the specified phase shift. Specifically, the controller 21 may calculate a further-corrected estimated value of the phase by adding the specified shift to the corrected estimated value 57 of the phase.

Accordingly, the information relating to the corrected estimated value 57 of the phase in step S205 may be constituted by information relating to the further-corrected estimated value of the phase. In the above-described situation where the operation of the controlled device is controlled, determining the drive amount of the controlled device from the corrected estimated value 57 of the phase may be determining the drive amount of the controlled device from the further-corrected estimated value of the phase. In the above-described situation where the gait assistance device 70 is used, determining the assistance amount 61 from the corrected estimated value 57 of the phase may be determining the assistance amount 61 from the further-corrected estimated value of the phase. In the above-described situation where the electrical stimulation device (functional electrical stimulation device 71) is used, determining the amount of electrical stimulation provided by the electrical stimulation device from the corrected estimated value 57 of the phase may be determining the amount of electrical stimulation provided by the electrical stimulation device from the further-corrected estimated value of the phase. In the above-described situation where the activation measurement device 73 is used, determining the amount of electrical stimulation in the activation measurement device 73 from the corrected estimated value 57 of the phase may be determining the amount of electrical stimulation in the activation measurement device 73 from the further-corrected estimated value of the phase. In the above-described situation where an abnormality in walking is detected, determining whether or not there is an abnormality in the walking of the user Z based on the corrected estimated value 57 of the phase may be determining whether or not there is an abnormality in the walking of the user Z based on the further-corrected estimated value of the phase.

Note that the timing for executing the processing of step S2041 need not be limited to the example of FIG. 20. The processing of step S2041 may be executed at any timing before step S2042. Also, in the example of FIG. 20, the processing of step S2041 is executed every time phase estimation is repeated. However, the processing of step S2041 does not necessarily need to be executed every time phase estimation is repeated. In another example, the processing of step S2041 may be executed every predetermined period of time, and the obtained phase shift value may be repeatedly used in the processing of step S2042 during the predetermined period.

### (Characteristics)

According to this modified example, the influence of delay can be reduced. As a result, in a situation where the controlled device is controlled, the operation of the controlled device (gait assistance device 70, functional electrical stimulation device 71, activation measurement device 73) can be controlled at more appropriate timing. In a situation where the gait assistance device 70 is controlled, another method for reducing the influence of delay is a method of correcting the assistance pattern 60 according to the delay, instead of making further corrections due to the phase shift. However, when this method is adopted, the influence of delay may vary depending on the user, making it difficult to compare assistance patterns to be provided between users. On the other hand, when the correction performed by the correction model 45 and the further correction performed due to the phase shift are employed, the assistance pattern 60 does not need to be changed, and therefore it is easier to perform comparison between users. Also, the assistance pattern 60 can be made into a template depending on the type of walking. Also, in a situation where an abnormality in walking is detected, it is possible to detect in real time whether or not there is an abnormality in walking.

<4.3>

In the above-described embodiment and modified examples, the correction model 45 may also be used regardless of the walking speed of the user Z. However, it was understood through a later-described experimental example that the error between the estimated value of the gait phase of the user calculated using the reference model and the ideal value can vary depending on the walking speed. In view of this, in the above-described embodiment and modified examples, the correction model 45 may also be generated according to the walking speed of the user Z. In one example, the correction model 45 may be generated for each reference speed such as 2 km/h, 3 km/h, and 4 km/h. The reference speed may be specified as a constant value or may be specified as a numerical value range. Note that, for convenience of description, the reference speed will be described below as being specified as a constant value.

As an example of how to use the plurality of generated correction models 45, in the estimation stage, the phase estimation device 2 (control devices 2A to 2C, monitoring device 2D) may measure the walking speed of the user Z and select one of the plurality of correction models 45 according to the measured walking speed. The phase estimation device 2 (control devices 2A to 2C, monitoring device 2D) may select, for example, the correction model 45 generated at the reference speed closest to the measured walking speed. Then, the phase estimation device 2 (control devices 2A to 2C, monitoring device 2D) may correct the estimated value 53 of the walking of the user Z using the selected correction model 45. As a result, the phase estimation device 2 (control devices 2A to 2C, monitoring device 2D) may acquire the corrected estimated value 57 of the phase.

In another example of the usage method, the phase estimation device 2 (control devices 2A to 2C, monitoring device 2D) may determine a compositing ratio of each of the plurality of correction models 45 according to the measured walking speed. As an example of the determination method, the phase estimation device 2 (control devices 2A to 2C, monitoring device 2D) may determine the compositing ratio of each correction model 45 such that the closer the reference speed is to the measured value of walking speed, the higher the compositing ratio of the corresponding correction model 45 is, and the farther the reference speed is from the measured value of the walking speed, the lower the compositing ratio of the corresponding correction model 45 is. Next, the phase estimation device 2 (control devices 2A to 2C, monitoring device 2D) may generate a composite correction model (hereinafter referred to as a “composite correction model”) by compositing the correction models 45 at the determined compositing ratios. The phase estimation device 2 (control devices 2A to 2C, monitoring device 2D) may correct the estimated value 53 of the walking of the user Z using the generated composite correction model. As a result, the phase estimation device 2 (control devices 2A to 2C, monitoring device 2D) may acquire the corrected estimated value 57 of the phase.

Note that the walking speed of the user Z may be measured by any sensor. The sensor S may be used to measure the walking speed of the user Z, or a sensor other than the sensor S may be used. When the sensor S is also used to measure walking speed, the information processing for measuring the walking speed may be at least partially common to the information processing for estimating the gait phase, or it may be completely separate from the information processing for estimating the gait phase. When the user Z walks on a treadmill, the speed of the treadmill may be used as the walking speed of the user Z. That is, the phase estimation device 2 (control devices 2A to 2C, monitoring device 2D) may obtain the measured value of the walking speed of the user Z from the treadmill. In another example, the amount of change in the gait phase may be proportional to the walking speed. In view of this, the phase estimation device 2 (control devices 2A to 2C, monitoring device 2D) may estimate the walking speed from the estimation result of the gait phase.

In yet another example, the faster the walking speed is, the greater the load acting on the soles of the user Z's feet is. In view of this, when using a plantar sensor, the phase estimation device 2 (control devices 2A to 2C, monitoring device 2D) may estimate the walking speed from the measured value of the plantar sensor. Alternatively, the load acting on the sole of the foot may be used instead of/in conjunction with the walking speed as an indicator for the correction model 45. That is, the correction model 45 may be generated according to the load acting on the soles of the user Z's feet. In this case, the plurality of generated correction models 45 may be used in the same manner as in the above-described walking speed example. In one example, the phase estimation device 2 (control devices 2A to 2C, monitoring device 2D) may select one of the plurality of correction models 45 according to the measured value of the plantar sensor. In another example, the phase estimation device 2 (control devices 2A to 2C, monitoring device 2D) may composite a plurality of correction models 45 according to the measured value of the plantar sensor.

### § 5 Experimental Example

In order to verify the effectiveness of the above-described embodiment, the following experiment was conducted. However, the present invention is not limited to the following experimental example.

In the experimental example, a correction model for the reference model was generated for each of two healthy adult males, and the gait phase on a treadmill was estimated. As a reference model (φ_{avg}), an estimator was used which was configured by a model obtained by approximating walking dynamics using the inverted pendulum cart model shown in Equations 1 to 4 below (Reference: J. Morimoto, G. Endo, J. Nakanishi, and G. Cheng. A Biologically Inspired Biped Locomotion Strategy for Humanoid Robots: Modulation of Sinusoidal Patterns by a Coupled Oscillator Model. IEEE Transactions on Robotics, 24 (1):185-191, February 2008).

$$[ Math . 1 ]$$

$${\dot{ϕ}}_{c} = {ω}_{c} + {K}_{c} sin ( {ϕ}_{a v g} - {ϕ}_{c} ) \qquad ( Equation 1 )$$

$$[ Math . 2 ]$$

$${\dot{ϕ}}_{a v g} = {ω}_{a v g} + {K}_{a v g} sin ( {ϕ}_{c} - {ϕ}_{a v g} ) \qquad ( Equation 2 )$$

Here, the left side of Equation 1 and the left side of Equation 2 are the gait phase estimated by the plantar sensor and the phase generated by the human walking dynamics. Also, ω_{c} (>0) and ω_{avg} (>0) are the human walking dynamics and the natural frequency of the estimator. K_{c} and K_{avg} are positive coupling constants. On the basis of this model, phase estimation using the reference model is possible using Equations 3 and 4 below.

$$[ Math . 3 ]$$

$$y = - ( {f}_{r} - {f}_{l} ) / ( {f}_{r} + {f}_{l} ) \qquad ( Equation 3 )$$

$$[ Math . 4 ]$$

$${ϕ}_{a v g} ( y ) = arctan ( \dot{y} / y ) \qquad ( Equation 4 )$$

Here, y is the center of pressure centered on a reference point on a floor of an inverted pendulum cart. fᵣ and fₗ are the left and right normalized vertical loads measured by the plantar sensors.

The relationship between the correction model (Østyle) and the reference model can be expressed by Equation 5 below.

$$[ Math . 5 ]$$

$${ϕ}_{style} = {ϕ}_{h} - {ϕ}_{a v g} \qquad ( Equation 5 )$$

Here, φₕ is the ideal value (true value) of the phase. In this experimental example, φₕ was obtained by causing each test subject to walk at a constant speed, dividing the obtained data by detecting the next heel contact from the heel contact, and normalizing the obtained data to the range from 0 to 2π. The correction model (φ_{style}) used the functions indicated by the following Equations 6 and 7.

$$[ Math . 6 ]$$

$${ϕ}_{style} ( ϕ ) = {\sum }_{j = 1}^{M} {ω}_{j} {ψ}_{j} ( ϕ ) \qquad ( Equation 6 )$$

$$[ Math . 7 ]$$

$${ψ}_{j} ( ϕ ) = {e}^{hcos ( ϕ - {c}_{j} ) - 1} \qquad ( Equation 7 )$$

M is the number of basis function divisions, and the larger M is, the more detailed fluctuations in the phase space can be tracked. In this experimental example, M was set to 25.

A force sensing resistor was used as a plantar sensor to estimate the gait phase. On the underside of the insole of each of left and right shoes, plantar sensors were disposed at positions corresponding to the heel and the ball of the foot (that is, a total of four plantar sensors were used). The sensor reading unit was fixed to the waist of the test subject with a belt, and each plantar sensor and the sensor reading unit were connected by wire. The sensor reading unit was connected to a personal computer using a LAN cable, and power was supplied via PoE (Power Over Ethernet).

In the experimental example, 16-bit AD conversion was performed on the measured value (FSR voltage) of the plantar sensor in the sensor reading unit. Then, sampling was performed using a personal computer with a control period of 250 Hz. A force sensing resistor has a property in which its resistance decreases non-linearly as the load increases. For this reason, fᵣ and fₗ were calculated by linearizing the measured value of each plantar sensor using a force sensing register model and normalizing the sum of the measured values of the heel and the ball of the foot. The treadmill was set up to enable external voltage control.

In the preparatory stage, in order to generate a correction model, the treadmill speed was set to 2 km/h (0.56 m/s), and data from the plantar sensor was obtained while walking was performed for 25 seconds. The timing of the start of measurement was controlled by each test subject, and therefore measurement of walking started in the middle of one gait cycle. Accordingly, a correction model for each test subject was generated by performing offline machine learning using the data excluding the data of the first cycle, so as to minimize the squared error.

In the estimation stage, the generated correction model was used to estimate the gait phase of each test subject in real time. The initial speed of the treadmill was set to 1 km/h (0.28 m/s). After a while, the speed of the treadmill was increased to 3 km/h (0.83 m/s). Thereafter, the speed of the treadmill was reduced to 2 km/h (0.56 m/s) and stopped. Each test subject maintained a walking position according to the speed of the treadmill and walked with a comfortable stride length. In the personal computer, phase estimation results (corrected estimated values) were calculated in real time at 250 Hz using the reference model and correction model, and the obtained estimated results were temporarily stored in the RAM. After one walking experiment was completed, the estimation result data stored in the RAM was stored in a storage. It was confirmed that it is possible to complete the processing from data sampling to gait phase estimation at 250 Hz (within 4 milliseconds) (i.e., it is possible to perform real-time analysis).

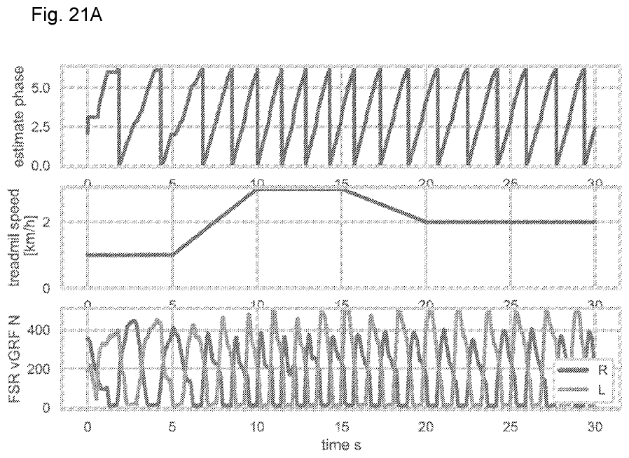

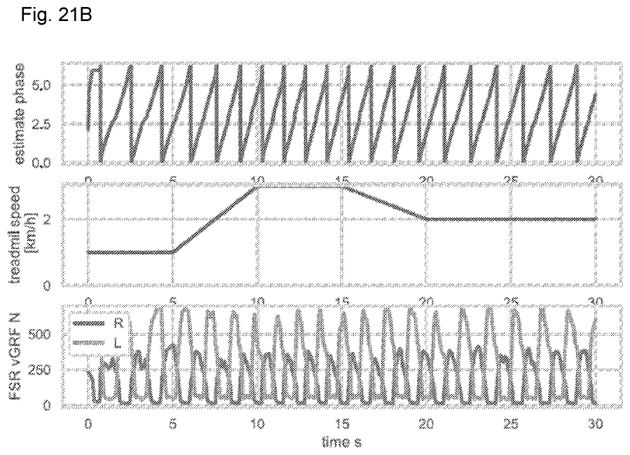

FIGS. 21A and 21B show the results of real-time phase estimation experiments performed on a first test subject and a second test subject. Specifically, FIG. 21A and FIG. 21B show, in order starting from the top, the phase estimation result, the speed of the treadmill, and the measured value of the plantar sensor (the estimated load value after linearization). As shown in FIGS. 21A and 21B, it was found that by using the correction model, the gait phase could be estimated easily and accurately in real time. It was also found that even if the walking speed fluctuates somewhat, the gait phase can be estimated accurately by using the correction model. Note that in this experimental example, the speed of the treadmill was set to 2 km/h (0.56 m/s), and a correction model was generated (calibrated). Accordingly, the phase estimation results were slightly distorted under conditions of different treadmill speeds. From this result, it was found that the error between the estimated value of the gait phase of the user calculated using the reference model and the ideal value can vary depending on the walking speed. In view of this, as described above, it has been estimated that by preparing a correction model according to the walking speed of the user, it is possible to estimate the gait phase of the user with higher accuracy.

### REFERENCE SIGNS LIST

1 Model generation device
11 Controller
12 Storage
13 Communication interface
14 External interface
15 Input device
16 Output device
17 Drive
81 Model generation program
91 Storage medium
111 Data acquisition unit
112 Phase estimation unit
113 Calculation unit
114 Generation unit
115 Evaluation unit
121 Reference model data
125 Correction model data
31 Sensor data
33 Estimated value
35 Ideal value
40 Reference model
45 Correction model
2 Phase estimation device
21 Controller
22 Storage
23 Communication interface
24 External interface
25 Input device
26 Output device
27 Drive
82 Phase estimation program
92 Storage medium
211 Acquisition unit
212 Phase estimation unit
213 Error estimation unit
214 Correction unit
215 Output unit
216 Monitoring unit
51 Sensor value
53 Estimated value
55 Error
57 Estimated value (of corrected phase)

1. A model generation method to be executed by a computer, comprising:
a step of acquiring sensor data generated by measuring walking of one or more cycles of a user with a sensor;
a step of calculating an estimated value of a gait phase in the acquired sensor data, using a reference model;
a step of calculating an ideal value of the gait phase corresponding to the calculated estimated value, based on a cycle of the walking that appears in the sensor data; and
a step of generating a correction model by modeling an error between the estimated value of the gait phase and the ideal value.

2. The model generation method according to claim 1, wherein
the sensor data is generated by measuring the walking of a plurality of cycles, and
after executing the step of calculating the estimated value of the phase, the computer further executes:
a step of calculating variation in the calculated estimated value of the phase; and
a step of performing notification of an alert if a magnitude of the variation in the estimated value exceeds a threshold.

3. The model generation method according to claim 1, wherein
the sensor data is generated by measuring the walking while the user receives assistance.

4. The model generation method according to claim 1, wherein
the computer repeatedly executes a generation cycle including the step of acquiring the sensor data, the step of calculating the estimated value of the phase, the step of calculating the ideal value of the phase, and the step of generating the correction model.

5. The model generation method according to claim 4, wherein
in the step of calculating the estimated value of the phase in the second generation cycle or later, the computer calculates the estimated value of the gait phase in the sensor data acquired in the current generation cycle by using a corrected reference model obtained by correcting a reference model used in the previous generation cycle using a correction model generated in the previous generation cycle.

6. The model generation method according to claim 4, wherein
after executing the generation cycle, the computer executes the next generation cycle in response to a request from an operator.

7. (canceled)

8. A phase estimation method to be executed by a computer, comprising:
a step of acquiring a sensor value of a sensor regarding walking of a user;
a step of calculating an estimated value of a gait phase from the acquired sensor value, using a reference model;
a step of estimating an error from the calculated estimated value of the phase, using a correction model;
a step of correcting the calculated estimated value of the phase using the estimated error; and
a step of outputting information relating to the corrected estimated value of the phase,
wherein the correction model is generated by modeling the error between the estimated value of the gait phase and an ideal value, using training sensor data generated by measuring the walking of one or more cycles of the user with the sensor.

9. The phase estimation method according to claim 8, wherein
the computer further executes:
a step of specifying a shift in the phase caused by a delay relating to estimation of the phase; and
a step of further correcting the corrected estimated value of the phase using the specified shift, and
information relating to the corrected estimated value of the phase is constituted by information relating to the further-corrected estimated value of the phase.

10. A control method to be executed by a computer, comprising:
a step of setting an assistance pattern;
a step of acquiring a sensor value of a sensor regarding walking of a user;
a step of calculating an estimated value of a gait phase from the acquired sensor value, using a reference model;
a step of estimating an error from the calculated estimated value of the phase, using a correction model;
a step of correcting the calculated estimated value of the phase using the estimated error;
a step of determining an assistance amount of a gait assistance device from the corrected estimated value of the phase in accordance with the set assistance pattern; and
a step of outputting the determined assistance amount,
wherein the correction model is generated by modeling the error between the estimated value of the gait phase and an ideal value, using training sensor data generated by measuring the walking of one or more cycles of the user with the sensor.

11. The control method according to claim 10, wherein
the assistance pattern is constituted by one or more muscle modules, and
the muscle module is configured by combining a plurality of periodic functions so as to reproduce muscular synergy.

12. The control method according to claim 10, wherein
the gait assistance device is configured to assist the walking through output of a pneumatic artificial muscle.

13. The control method according to claim 10, wherein
the computer further executes:
a step of specifying a shift in the phase caused by a delay relating to estimation of the phase; and
a step of further correcting the corrected estimated value of the phase using the specified shift, and
determining the assistance amount from the corrected estimated value of the phase is determining the assistance amount from the further-corrected estimated value of the phase.

14. The control method according to claim 10, wherein
the computer repeatedly executes an estimation cycle including the step of acquiring the sensor value, the step of calculating the estimated value of the phase, the step of estimating the error, the step of correcting the estimated value of the phase, the step of determining the assistance amount, and the step of outputting the assistance amount, and
in response to repeatedly executing the estimation cycle for the walking of one or more cycles of the user, the computer further executes:
a step of calculating an ideal value of the gait phase with respect to the corrected estimated value, based on a cycle of the walking;
a step of calculating an error between the corrected estimated value and the ideal value; and
a step of outputting information relating to the calculated error.

15. The control method according to claim 10, wherein
the computer repeatedly executes an estimation cycle including the step of acquiring the sensor value, the step of calculating the estimated value of the phase, the step of estimating the error, the step of correcting the estimated value of the phase, the step of determining the assistance amount, and the step of outputting the assistance amount, and
in the step of determining the assistance amount in the second estimation cycle or later, the computer
calculates an amount of change between the corrected estimated value in the previous estimation cycle and the corrected estimated value in the current estimation cycle,
determines whether or not the calculated amount of change satisfies a permissible condition,
if the amount of change satisfies the permissible condition, determines an assistance amount in the current estimation cycle from the corrected estimated value in the current estimation cycle, and
if the amount of change does not satisfy a permissible condition, determines the assistance amount in the current estimation cycle based on the corrected estimation value in the previous estimation cycle, without relying on the corrected estimation value in the current estimation cycle.

16-17. (canceled)
